(19) United States
(12) Patent Application Publication
(10) Pub. No.: US 2023/0377398 A1
(43) Pub. Date: Nov. 23, 2023

(54) INFORMATION PROCESSING APPARATUS, INFORMATION PROCESSING METHOD, AND STORAGE MEDIUM

(71) Applicant: NEC Corporation, Minato-ku, Tokyo (JP)

(72) Inventors: Takumi OTANI, Tokyo (JP); Takeshi SASAMOTO, Tokyo (JP); Junichi INOUE, Tokyo (JP)

(73) Assignee: NEC Corporation, Minato-ku, Tokyo (JP)

(21) Appl. No.: 18/030,824

(22) PCT Filed: Oct. 9, 2020
(86) PCT No.: PCT/JP2020/038385

§ 371 Date: Apr. 7, 2023

(51) Int. Cl. G07C 9/37; G16H 50/80; A61B 5/00; G06V 40/16

(52) U.S. Cl. CPC G07C 9/37; G16H 50/80; A61B 5/0008; G06V 40/172

(57) ABSTRACT

An information processing apparatus according to the present invention includes: an acquisition unit that acquires body surface temperatures of a user of an airplane measured at procedure places of a plurality of procedures imposed on the user at a departure airport; and a determination unit that determines whether the user is allowed to board the airplane based on the plurality of body surface temperatures acquired from the user.

### TECHNICAL FIELD

The present invention relates to an information processing apparatus, an information processing method, and a storage medium.

### BACKGROUND ART

Patent Literature 1 discloses a risk determination system in which an inspection terminal is installed at a gate that controls the entry and exit of passengers. The inspection terminal of the risk determination system acquires information on the degree of risk that the user is suffering from a disease from a risk determination device based on location information and biometric information (body temperature, pulse rate, etc.) acquired from a wearable terminal worn by the passenger, and displays the acquired information on a screen.

### CITATION LIST

### Patent Literature

PTL 1: Japanese Patent Application Laid-Open No. 2016-195639

### SUMMARY OF INVENTION

### Technical Problem

According to the system described in Patent Literature 1, when a user (passenger) who has passed through a specific gate is in a fever state, the user can be detected as a person of high risk. However, the health state of the user can change significantly in time from the time the user arrives at the airport to the time of actually boarding the airplane. Thus, for example, if the health status of a user passing through the specific gate is temporarily improving, there is a risk that a person with an actual health problem will be allowed to board the airplane.

Accordingly, the present invention has been made in view of such circumstances and intends to provide an information processing apparatus, an information processing method, and a storage medium that can efficiently detect a person with a health problem at a departure airport.

### Solution to Problem

According to one aspect of the present invention, provided is an information processing apparatus including: an acquisition unit that acquires body surface temperatures of a user of an airplane measured at procedure places of a plurality of procedures imposed on the user at a departure airport; and a determination unit that determines whether the user is allowed to board the airplane based on the plurality of body surface temperatures acquired from the user.

According to another aspect of the present invention, provided is an information processing method including: acquiring body surface temperatures of a user of an airplane measured at procedure places of a plurality of procedures imposed on the user at a departure airport; and determining whether the user is allowed to board the airplane based on the plurality of body surface temperatures acquired from the user.

According to yet another aspect of the present invention, provided is a storage medium storing a program that causes a computer to perform: acquiring body surface temperatures of a user of an airplane measured at procedure places of a plurality of procedures imposed on the user at a departure airport; and determining whether the user is allowed to board the airplane based on the plurality of body surface temperatures acquired from the user.

### Advantageous Effects of Invention

According to the present invention, an information processing apparatus, an information processing method, and a storage medium that can efficiently detect a person with a health problem at a departure airport.

### BRIEF DESCRIPTION OF DRAWINGS

FIG. 1 is a block diagram illustrating an example of an overall configuration of an information processing system according to a first example embodiment.

FIG. 2 is a diagram illustrating an example of information stored in a token ID information DB according to the first example embodiment.

FIG. 3 is a diagram illustrating an example of information stored in a passage history information DB according to the first example embodiment.

FIG. 4 is a diagram illustrating an example of information stored in an operation information DB according to the first example embodiment.

FIG. 5 is a block diagram illustrating an example of a hardware configuration of a management server according to the first example embodiment.

FIG. 6 is a block diagram illustrating an example of a hardware configuration of a check-in terminal according to the first example embodiment.

FIG. 7 is a block diagram illustrating an example of a hardware configuration of an automatic baggage drop-off machine according to the first example embodiment.

FIG. 8 is a block diagram illustrating an example of a hardware configuration of a security inspection apparatus according to the first example embodiment.

FIG. 9 is a block diagram illustrating an example of a hardware configuration of an automated gate apparatus according to the first example embodiment.

FIG. 10 is a block diagram illustrating an example of a hardware configuration of a boarding gate apparatus according to the first example embodiment.

FIG. 11 is a block diagram illustrating an example of a hardware configuration of an operation terminal according to the first example embodiment.

FIG. 12 is a sequence chart illustrating an example of a process in a check-in procedure performed by the information processing system according to the first example embodiment.

FIG. 13 is a diagram illustrating a state where a face image and a thermography image are captured at the check-in terminal according to the first example embodiment.

FIG. 14 is a sequence chart illustrating an example of a process in an automatic baggage drop-off procedure performed by the information processing system according to the first example embodiment.

FIG. 15 is a sequence chart illustrating an example of a process in a security inspection procedure performed by the information processing system according to the first example embodiment.

FIG. 16 is a sequence chart illustrating an example of a process in a departure inspection procedure performed by the information processing system according to the first example embodiment.

FIG. 17 is a sequence chart illustrating an example of a process in an identity verification procedure before boarding performed by the management server according to the first example embodiment.

FIG. 18A is a diagram illustrating an example of a screen displayed on the boarding gate apparatus according to the first example embodiment.

FIG. 18B is a diagram illustrating an example of a screen displayed on the boarding gate apparatus according to the first example embodiment.

FIG. 18C is a diagram illustrating an example of a screen displayed on the boarding gate apparatus according to the first example embodiment.

FIG. 19 is a diagram illustrating an example of a screen displayed on the operation terminal according to the first example embodiment.

FIG. 20 is a flowchart illustrating an example of a process for determining whether or not to allow boarding in the management server according to the first example embodiment.

FIG. 21 is a diagram illustrating an example of measurement history information on body surface temperatures for each user measured at each touch point according to the first example embodiment.

FIG. 22 is a sequence chart illustrating an example of a process in a security inspection procedure of the information processing system according to a second example embodiment.

FIG. 23 is a flowchart illustrating an example of a process for determining whether or not to allow boarding in the management server according to the second example embodiment.

FIG. 24 is a flowchart illustrating an example of a process for determining whether or not to allow boarding in the management server according to a third example embodiment.

FIG. 25 is a flowchart illustrating an example of a process for determining whether or not to allow boarding in the management server according to a fourth example embodiment.

FIG. 26 is a flowchart illustrating an example of a process for determining whether or not to allow boarding in the management server according to a fifth example embodiment.

FIG. 27 is a function block diagram of an information processing apparatus according to a sixth example embodiment.

### DESCRIPTION OF EMBODIMENTS

Exemplary example embodiments of the present invention will be described below with reference to the drawings. Throughout the drawings, similar elements or corresponding elements are labeled with the same references, and the description thereof may be omitted or simplified.

### First Example Embodiment

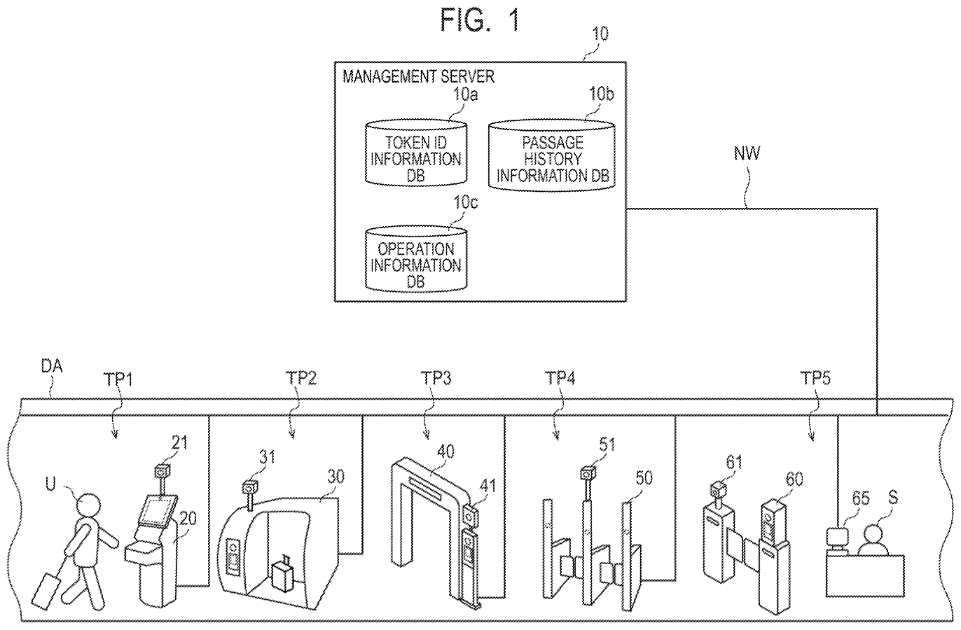

FIG. 1 is a schematic diagram illustrating an example of the overall configuration of an information processing system according to the present example embodiment. The information processing system according to the present example embodiment is a computer system that supports a series of procedures performed on a user U in a first country and a second country, respectively, when the user U departs from the first country at an airport DA of the first country and enters the second country at an airport of the second country by an airplane. The information processing system is run by a public institution such as an immigration control bureau or a trustee entrusted with the operation from such an institution, for example.

As illustrated in FIG. 1, the information processing system includes management servers 10, a check-in terminal 20, an automatic baggage drop-off machine 30, a security inspection apparatus 40, an automated gate apparatus 50, a boarding gate apparatus 60, and an operation terminal 65. The management server 10 is connected to apparatuses in the airport DA via networks NW, respectively. The networks NW, is each formed of a Wide Area Network (WAN) such as the Internet or a Local Area Network (LAN). The connection scheme may be a wireless scheme without being limited to a wired scheme.

The management servers 10 each are an information processing apparatus that manages various procedures on the user U during entry to or departure from countries. The management server 10 is installed in a facility of an airport company, an airline company, or the like, for example. Note that the management server 10 is not required to be a single server and may be configured as a server group including a plurality of servers. Further, the management server 10 is not necessarily required to be provided on a country basis and may be configured as a server used by a plurality of countries in a shared manner.

The management server 10 performs identity verification on the user U by matching a face image captured by the check-in terminal 20, which is a face authentication terminal, with a passport face image read from a passport by the check-in terminal 20.

Furthermore, the management server 10 performs identity verification on the user U by matching a face image captured by another face authentication terminal (each of the automatic baggage drop-off machine 30, the security inspection apparatus 40, the automated gate apparatus 50, the boarding gate apparatus 60, or the like) with a registered face image registered in a database, respectively.

Further, as illustrated in FIG. 1, the management server 10 includes a token ID information DB 10a, a passage history information DB 10b, and an operation information DB 10c. These databases are examples, and the management server 10 may further include other databases. Further, a plurality of databases may be aggregated into a single database.

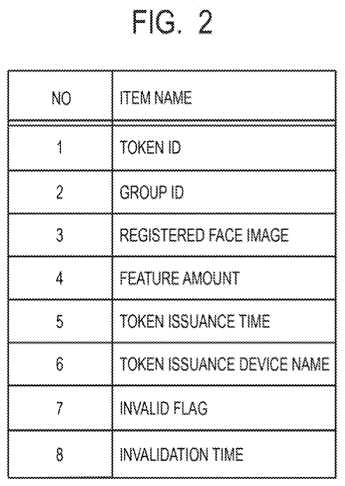

FIG. 2 is a diagram illustrating an example of information stored in the token ID information DB 10a. The token ID information DB 10a has data items of a token ID, a group ID, a registered face image, a feature amount, a token issuance time, a token issuance device name, an invalid flag, and an invalidation time.

The token ID is an identifier that uniquely identifies ID information. The token ID of the present example embodiment is issued by the management server 10 provided that a result of a matching process is that the matching is successful where the matching process is to match a captured face image, which is obtained by the user U capturing his/her face by himself/herself by using a face authentication terminal such as the check-in terminal 20, with a passport face image of the user U read from a passport by the face authentication terminal. Further, for example, after the user U finishes the travel from the first country to the second country, the token ID is invalidated. That is, a token ID is not an identifier used permanently but a onetime ID having a validity period (lifecycle).

Note that the term “matching is successful” in the present example embodiment means that a matching score indicating a similarity between biometric information on the user U and registered biometric information on a registrant is greater than or equal to a predetermined threshold. In contrast, the term “matching is unsuccessful” means that a matching score is less than the predetermined threshold.

The group ID is an identifier for grouping ID information. The registered face image is a face image registered for the user U. In the present example embodiment, a face image of the user U captured during the initial procedure in the airport DA of the first country or a passport face image read from an IC chip of a passport of the user U by a reading device is used as a registered face image stored in the token ID information DB 10a. The feature amount is a value extracted from biometric information (registered face image).

Further, although the term of biometric information in the present example embodiment means a face image and a feature amount extracted from the face image, the biometric information is not limited to a face image and a face feature amount. That is, biometric authentication may be performed by using an iris image, a fingerprint image, a palmprint image, an auricular image, or the like as biometric information on the user U.

The token issuance time is a time that the management server 10 issued a token ID. The token issuance device name is a device name from which a registered face image which triggered issuance of a token ID is acquired. The invalid flag is flag information indicating whether or not a token ID is currently valid. For example, upon issuance of a token ID, the invalid flag is set to a value indicating a state where the token ID is valid. Further, in response to satisfying a predetermined condition, the invalid flag is updated to a value indicating a state where a token ID is invalid. The invalidation time is a timestamp indicating a time the invalid flag is invalidated.

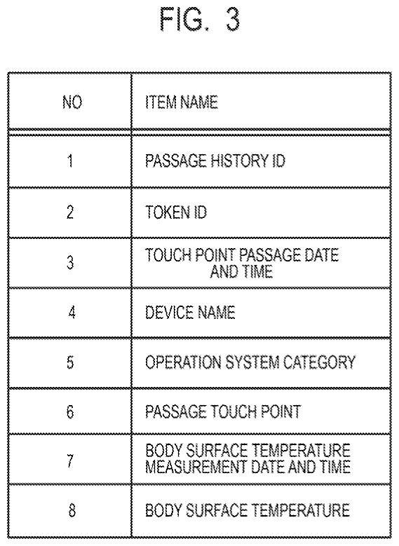

FIG. 3 is a diagram illustrating an example of information stored in the passage history information DB 10b. The passage history information DB 10b has data items of a passage history ID, a token ID, a touch point passage date and time, a device name, an operation system category, a passage touch point, a body surface temperature measurement date and time, and a body surface temperature. The passage history ID is an identifier that uniquely identifies passage history information. The touch point passage date and time is a timestamp indicating a time the user U passes through a touch point. The device name is a machine name of an operation terminal used for a procedure at a touch point. The operation system category is a category of an operation system which an operation terminal belongs to. The passage touch point is a name of a touch point through which the user U passes. The body surface temperature measurement date and time is a timestamp when a body surface temperature of the user U is measured by capturing of a thermography image. The body surface temperature is a temperature measured for a skin surface of the user U.

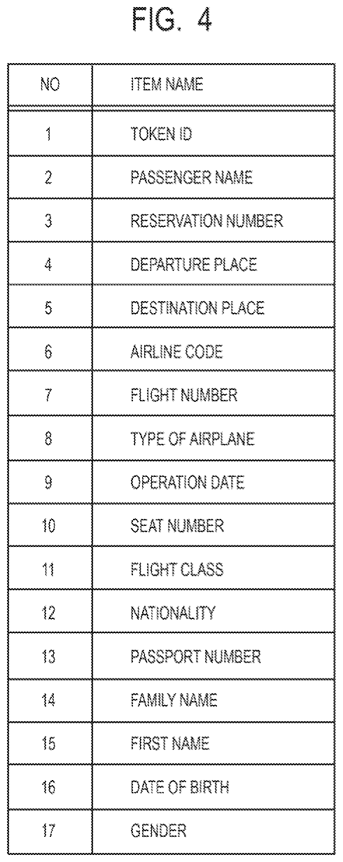

FIG. 4 is a diagram illustrating an example of information stored in the operation information DB 10c. The operation information DB 10c has data items of a token ID, a passenger name, a reservation number, a departure place, a destination place, an airline code, a flight number, a type of airplane, an operation date, a seat number, a flight class, a nationality, a passport number, a family name, a first name, a date of birth, and a gender.

The reservation number is an identifier that uniquely identifies boarding reservation information. The airline code is an identifier that uniquely identifies an airline company. The flight class is a class of a seat and may be, for example, first class, business class, economy class, or the like. In general, a seat of a higher flight class has a longer distance to the next seat and a longer distance (seat pitch) to the front and rear seats. Further, services that the user U may receive in an airport and a cabin are also different in accordance with a flight class.

Information on a passenger name, a reservation number, a departure place, a destination place, an airline code, a flight number, a type of an airplane, an operation date, a seat number, a nationality, a passport number, a family name, a first name, a date of birth, a gender, or the like may be acquired from a medium such as a passport and a boarding ticket or acquired from a database that manages reservation information (not illustrated) by using passport number, a reservation number, or the like as a key.

In such a way, the operation information DB 10c stores operation information about a predetermined operation in association with a token ID. In the present example embodiment, “predetermined operation” means a procedure operation (check-in/baggage drop-off/security inspection/departure inspection/identity verification on a passenger, or the like) performed at each touch point in an airport.

Next, the apparatuses responsible for procedural operations on the user U in cooperation with the management server 10 in the airport DA according to the present example embodiment will be described.

The check-in terminal 20 is installed in a check-in lobby or a check-in counter in each of the airport DA. Hereafter, the procedural area where the check-in terminal 20 is installed is referred to as “touch point TP1”. The check-in terminal 20 is a self-service terminal operated by the user U by himself/herself to perform a check-in procedure (a boarding procedure). After completion of the check-in procedure at the touch point TP1, the user U proceeds to a baggage drop-off place or a security inspection site.

The automatic baggage drop-off machine 30 is installed in a region adjacent to a baggage counter (a manned counter) or a region near the check-in terminal 20 in each of the airport DA. Hereafter, the procedural area where the automatic baggage drop-off machine 30 is installed is referred to as “touch point TP2”. The automatic baggage drop-off machine 30 is a self-service terminal operated by the user U by himself/herself to perform a procedure to drop off, to an airline company, baggage not to be carried in the cabin. After completion of the baggage drop-off procedure at the touch point TP2, the user U proceeds to the security inspection site. When the user U does not drop off his/her baggage, the procedure at the touch point TP2 is omitted.

The security inspection apparatus 40 is installed in the security inspection site (hereafter, referred to as “touch point TP3”) in each of the airport DA. The term “security inspection apparatus” in the present example embodiment is used as a meaning including all of a metal detector that checks whether or not the user U is wearing a metal item that may be a dangerous object, an X-ray inspection device that uses an X-ray to check whether or not a dangerous object is included in carry-on baggage or the like, a passage control device that determines whether or not to permit passage of the user U at an entrance or an exit of the security inspection site, and the like. After completion of the security inspection procedure at the touch point TP3, the user U proceeds to a departure inspection site.

The automated gate apparatus 50 is installed at the departure inspection site (hereafter, referred to as “touch point TP4”) in each of the airport DA. The automated gate apparatus 50 is an apparatus that automatically performs a departure inspection procedure on the user U. After completion of the departure inspection procedure at the touch point TP4, the user U proceeds to a departure area where a duty-free shop and a boarding gate are provided.

The boarding gate apparatus 60 is installed to each boarding gate (hereafter, referred to as “touch point TP5”) in each of the airport DA. The boarding gate apparatus 60 is a passage control apparatus that checks whether or not the user U is a passenger of an airplane associated with the boarding gate. After completion of the procedure at the touch point TP5, the user U boards the airplane and departs to the second country. In such a way, the check-in terminal 20, the automatic baggage drop-off machine 30, the security inspection apparatus 40, the automated gate apparatus 50, and the boarding gate apparatus 60 are used when the user U departs from the first country.

The operation terminal 65 installed to a manned counter of boarding gate in the airport DA. The operation terminal 65 is a terminal used by a staff member S of an airline company or an airport facility for his/her operation. The operation terminal 65 is a personal computer, a tablet terminal, a smartphone, or the like, for example. Note that, although only the operation terminal 65 at the touch point TP5 is depicted for simplified illustration in the present example embodiment, such operation terminals may be installed similarly at other touch points.

Also, as illustrated in FIG. 1, the check-in terminal 20, the automatic baggage drop-off machine 30, the security inspection apparatus 40, the automated gate apparatus 50 and the boarding gate apparatus 60 include thermography devices 21, 31, 41, 51 and 61, respectively. Herein, the thermography device 21 is described as a representative example.

The thermography device 21 is an image capturing device that analyzes infrared rays emitted from an object and generates a thermography image representing a heat distribution. The thermography device 21 has the following advantages.
(A) It is possible to measure a body surface temperature in a contactless manner with an object to be measured.
(B) It is possible to visualize, as an image, a temperature distribution of a wide area as a plane rather than a temperature value at a point on an object.
(C) It is possible to measure a body surface temperature in real time because the response speed is high.

Because of such advantages, the thermography device 21 according to the present example embodiment is used for measuring the body surface temperature of the user U in the airport DA.

Next, a hardware configuration of devices forming the information processing system will be described. Note that, throughout a plurality of drawings, devices having the same name and differing only in the reference are devices having substantially the same function, and thus, the detailed description thereof will be omitted in the subsequent drawings.

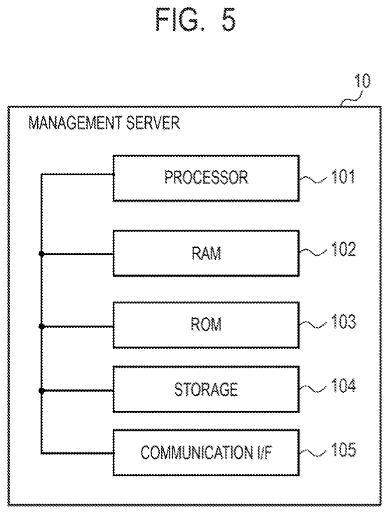

FIG. 5 is a block diagram illustrating an example of a hardware configuration of the management server 10. The management server 10 includes a processor 101, a random access memory (RAM) 102, a read only memory (ROM) 103, a storage 104, and a communication interface (I/F) 105, as a computer that performs calculation, control, and storage. These devices are connected to each other via a bus, a wiring, a drive device, or the like.

The processor 101 has functions of performing predetermined calculation in accordance with a program stored in the ROM 103, the storage 104, or the like and controlling each unit of the management server 10. Further, as the processor 101, one of a central processing unit (CPU), a graphics processing unit (GPU), a field programmable gate array (FPGA), a digital signal processor (DSP), and an application specific integrated circuit (ASIC) may be used, or a plurality thereof may be used in parallel.

The RAM 102 is formed of a volatile storage medium and provides a temporary memory area required for the operation of the processor 101. The ROM 103 is formed of a nonvolatile storage medium and stores information required such as a program used for the operation of the management server 10.

The storage 104 is formed of a nonvolatile storage medium and performs storage of a database, storage of an operating program of the management server 10, or the like. The storage 104 is formed of a hard disk drive (HDD) or a solid state drive (SSD), for example.

The communication I/F 105 is a communication interface based on a specification such as Ethernet (registered trademark), Wi-Fi (registered trademark), 4G, or the like and is a module for communicating with other devices.

The processor 101 loads a program stored in the ROM 103, the storage 104, or the like into the RAM 102 and executes the program to perform a predetermined calculation process. Further, the processor 101 controls each unit of the management server 10, such as the communication I/F 105, based on the program.

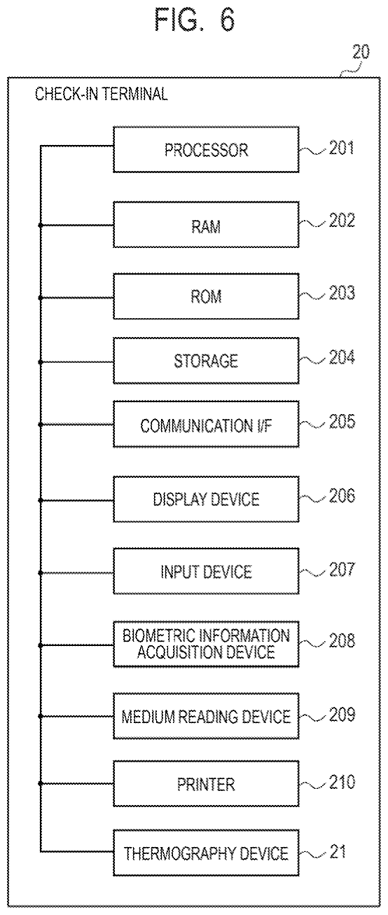

FIG. 6 is a block diagram illustrating an example of the hardware configuration of the check-in terminal 20. The check-in terminal 20 includes a processor 201, a RAM 202, a ROM 203, a storage 204, a communication I/F 205, a display device 206, an input device 207, a biometric information acquisition device 208, a medium reading device 209, a printer 210, and the thermography device 21. These devices are connected to each other via a bus, a wiring, a drive device, or the like.

The display device 206 is a liquid crystal display, an organic light emitting diode (OLED) display, or the like configured to display a moving image, a static image, a text, or the like and is used for presenting information to the user U.

The input device 207 is a keyboard, a pointing device, a button, or the like and accepts a user operation. The display device 206 and the input device 207 may be formed integrally as a touch panel.

The biometric information acquisition device 208 is a device that acquires a face image of the user U as biometric information on the user U. The biometric information acquisition device 208 is a digital camera having a Complementary Metal-Oxide-Semiconductor (CMOS) image sensor, a Charge Coupled Device (CCD) image sensor, or the like as a light receiving element, for example. The biometric information acquisition device 208 captures an image of a face of the user U standing in front of the device to acquire the face image, for example.

The medium reading device 209 is a device that reads information recorded or stored in a medium carried by the user U. The medium reading device 209 may be, for example, a code reader, an image scanner, a contactless integrated circuit (IC) reader, an optical character reader (OCR) device, or the like. Further, a recording medium or a storage medium may be, for example, a paper airline ticket, a mobile terminal displaying a receipt of an e-ticket, or the like. The printer 210 prints a boarding ticket in which boarding information and guidance information about procedures up to boarding are printed at the time of completion of a check-in procedure.

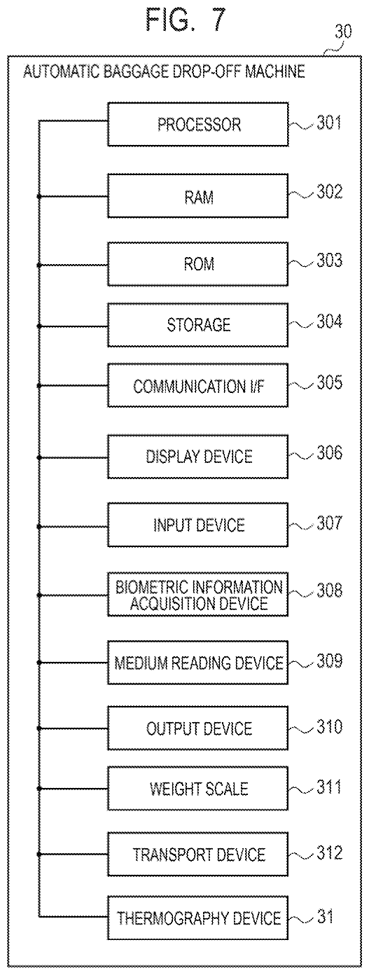

FIG. 7 is a block diagram illustrating an example of a hardware configuration of an automatic baggage drop-off machine 30 according to the present example embodiment. The automatic baggage drop-off machine 30 includes a processor 301, a RAM 302, a ROM 303, a storage 304, a communication I/F 305, a display device 306, an input device 307, a biometric information acquisition device 308, a medium reading device 309, a output device 310, a weight scale 311, a transport device 312, and the thermography device 31. These devices are connected to each other via a bus, a wiring, a drive device, or the like.

The output device 310 is a device that outputs a baggage tag attached to checked baggage. For example, the baggage tag is an RFID tag having an IC chip that stores tag information including a checked baggage ID, a token ID, a flight number, or the like. Further, the output device 310 further outputs a baggage claim tag required for claiming checked baggage after arriving at the destination. The baggage tag or the baggage claim tag is associated with at least one of a reservation number, a boarding ticket number, and a token ID, for example.

The weight scale 311 measures the weight of checked baggage and outputs a measured value to the processor 301. When the weight of checked baggage exceeds a predetermined threshold, the processor 301 outputs error information that urges the user U to take some action. The transport device 312 transports checked baggage on a receiving area placed by the user U.

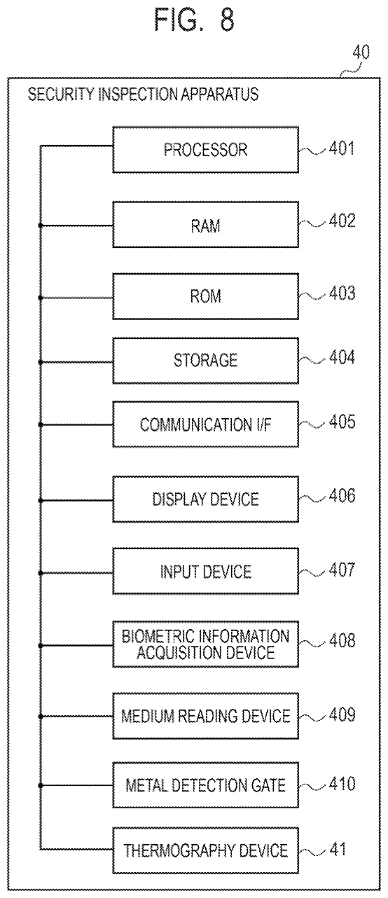

FIG. 8 is a block diagram illustrating an example of a hardware configuration of a security inspection apparatus 40 according to the present example embodiment. The security inspection apparatus 40 includes a processor 401, a RAM 402, a ROM 403, a storage 404, a communication I/F 405, a display device 406, an input device 407, a biometric information acquisition device 408, a medium reading device 409, a metal detection gate 410, and the thermography device 41. These devices are connected to each other via a bus, a wiring, a drive device, or the like.

The metal detection gate 410 is a gate-type metal detector and detects a metal item worn by a user U passing through the metal detection gate 410.

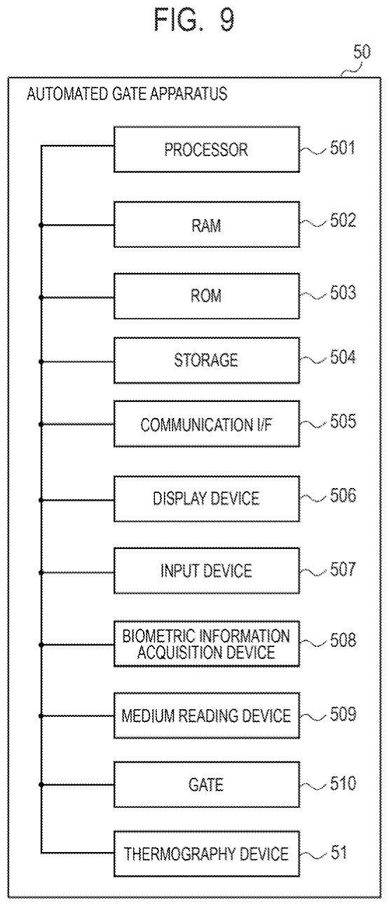

FIG. 9 is a block diagram illustrating an example of a hardware configuration of the automated gate apparatus 50 according to the present example embodiment. The automated gate apparatus 50 includes a processor 501, a RAM 502, a ROM 503, a storage 504, a communication I/F 505, a display device 506, an input device 507, a biometric information acquisition device 508, a medium reading device 509, a gate 510, and a thermography device 51. These devices are connected to each other via a bus, a wiring, a drive device, or the like.

The gate 510 transitions from a closed state to block passage of the user U during standby to an open state to permit passage of the user U under the control of the processor 501 when identity verification of the user U at the automated gate apparatus 50 is successful. The type of the gate 510 is not particularly limited and may be, for example, a flapper gate in which one or more flappers provided to one side or both sides of a passage are opened and closed, a turn style gate in which three bars are revolved, or the like.

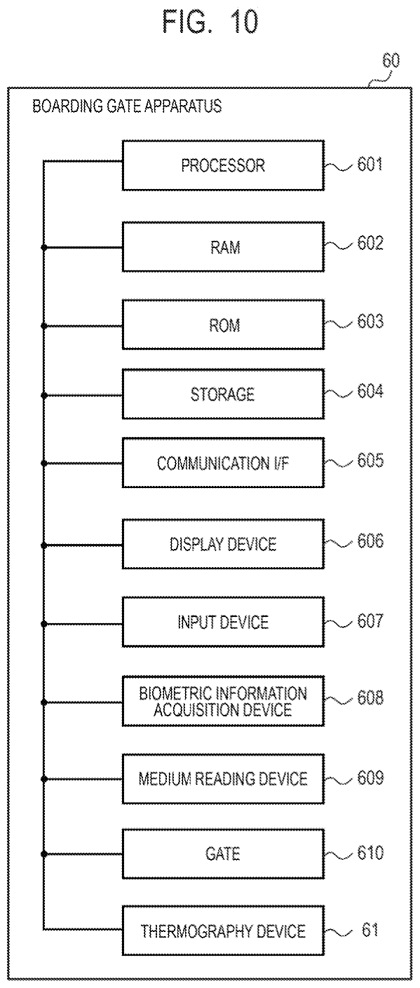

FIG. 10 is a block diagram illustrating an example of the hardware configuration of the boarding gate apparatus 60. The boarding gate apparatus 60 includes a processor 601, a RAM 602, a ROM 603, a storage 604, a communication I/F 605, a display device 606, an input device 607, a biometric information acquisition device 608, a medium reading device 609, a gate 610, and a thermography device 61. These devices are connected to each other via a bus, a wiring, a drive device, or the like.

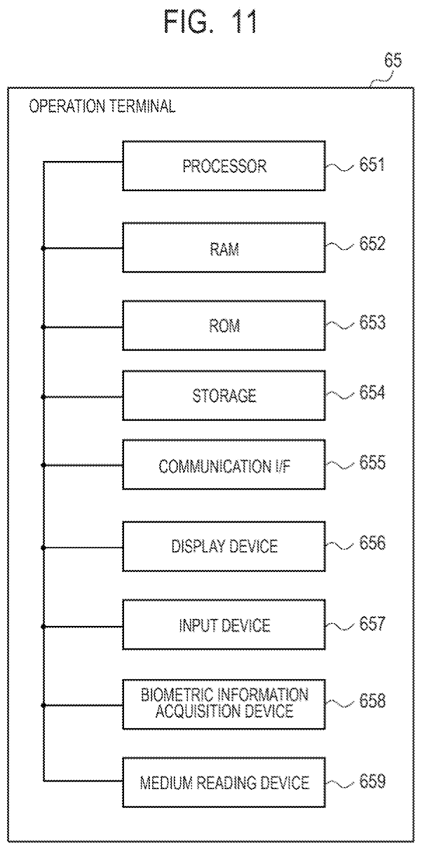

FIG. 11 is a block diagram illustrating an example of a hardware configuration of an operation terminal 65 according to the present example embodiment. The operation terminal 65 includes a processor 651, a RAM 652, a ROM 653, a storage 654, a communication I/F 655, a display device 656, an input device 657, a biometric information acquisition device 658, and a medium reading device 659. These devices are connected to each other via a bus, a wiring, a drive device, or the like.

Note that the hardware configurations illustrated in FIG. 5 to FIG. 11 are examples, a device other than the above may be added, and some of the devices may be omitted. Further, some of the devices may be replaced with another device having the same function. Further, some of the functions of the present example embodiment may be provided by another device via a network, or the functions of the present example embodiment may be distributed to and implemented by a plurality of devices. In such a way, the hardware configurations illustrated in FIG. 5 to FIG. 11 can be changed as appropriate.

Next, the operation of the apparatuses in the information processing system according to the present example embodiment will be described with reference to the drawings.

### [Check-in Procedure]

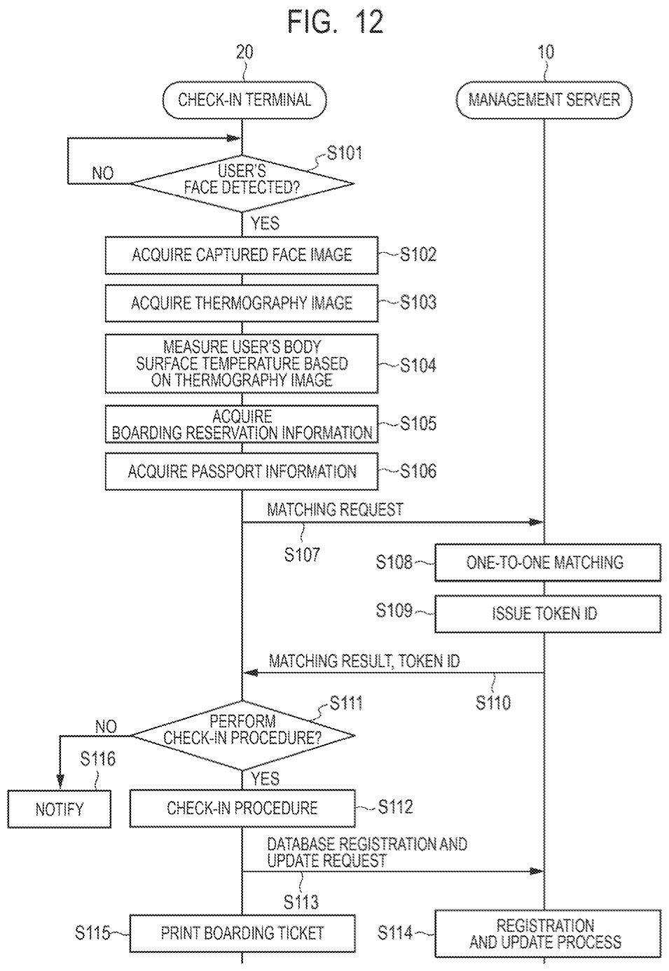

FIG. 12 is a sequence chart illustrating an example of the process in a check-in procedure of the information processing system according to the present example embodiment.

First, the check-in terminal 20 captures an image of the area in front thereof constantly or periodically and determines whether or not a face of a user U standing in front of the check-in terminal 20 is detected in the captured image (step S101). The check-in terminal 20 stands by until a face of a user U is detected in the image by the biometric information acquisition device 208 (step S101: NO).

If the check-in terminal 20 determines that a face of a user U is detected by the biometric information acquisition device 208 (step S101: YES), the check-in terminal 20 captures an image of the face of the user U and acquires the captured face image of the user U as a target face image (step S102). Note that it is preferable to display a window for obtaining user U consent before capturing a face image.

Next, the check-in terminal 20 captures an image of the face of the user U by the thermography device 21 and acquires a thermography image (step S103). That is, the check-in terminal 20 captures a thermography image in synchronization with capturing of a captured face image.

Next, the check-in terminal 20 measures the body surface temperature of the user U based on the thermography image (step S104).

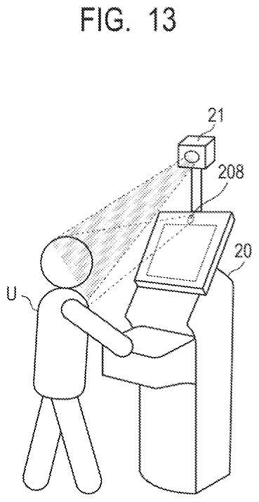

FIG. 13 is a diagram illustrating a state where a face image and a thermography image are captured at the check-in terminal 20. This illustrates an example in which a thermography image including the face of the user U is captured by the thermography device 21 while the face image of the user U is being captured by the biometric information acquisition device 208. It is preferable for the thermography device 21 to start image capturing in response to a timing of capturing performed by the biometric information acquisition device 208 rather than continuously capturing thermography images. This makes it possible to further associate thermography images with a token ID of the user U identified from the face image.

Next, in response to an airline ticket medium being held over the reading area of the medium reading device 209, the check-in terminal 20 acquires boarding reservation information on the user U from the airline ticket medium held over (step S105). The boarding reservation information includes attribute information on the user U (a family name, a first name, a gender, or the like) or flight information (an airline code, a flight number, a boarding date, a departure place, a transit point, a destination place, a seat number, a departure time, an arrival time, or the like).

Next, when a passport is held over the reading area of the medium reading device 209, the check-in terminal 20 acquires passport information on the user U from the passport held over (step S106). The passport information includes a passport face image of the user U, identity verification information, a passport number, information on a country that has issued the passport, or the like.

Next, the check-in terminal 20 requests the management server 10 to match face images (step S107). The data of the matching request includes a captured face image captured at the current place and the passport face image read from the passport.

In response to receiving information from the check-in terminal 20, the management server 10 performs one-to-one matching between the captured face image captured by the check-in terminal 20 and the passport face image (step S108).

Next, the management server 10 issues a token ID provided that the matching result in step S108 is that the matching is successful (step S109) and transmits the matching result and the token ID to the check-in terminal 20 (step S110).

Next, based on the matching result received from the management server 10, the check-in terminal 20 determines whether or not a check-in procedure for the user U is ready to be performed (step S111).

In this step, if the check-in terminal 20 determines that the check-in procedure for the user U is not ready to be performed (step S111: NO), the check-in terminal 20 notifies the user U of an error message (step S116) and ends the process.

In contrast, if the check-in terminal 20 determines that the matching result at the management server 10 is that the matching is successful and determines that the check-in procedure for the user U is ready to be performed (step S111: YES), the check-in terminal 20 performs a check-in procedure such as confirmation of an itinerary, selection of a seat, or the like based on input information from the user U (step S112). In response to completion of the check-in procedure, the check-in terminal 20 transmits a database registration and update request to the management server 10 (step S113).

Next, in response to receiving the database registration and update request from the check-in terminal 20, the management server 10 performs a registration process and an update process on the passage history information DB 10b and the operation information DB 10c (step S114). Specifically, the passage history information at the touch point TP1 and measurement history information on user U's body surface temperature is registered to the passage history information DB 10b in association with the token ID.

The check-in terminal 20 then prints a boarding ticket describing boarding reservation information and guidance information about procedures up to boarding (step S115) and ends the process.

### [Baggage Drop-Off Procedure]

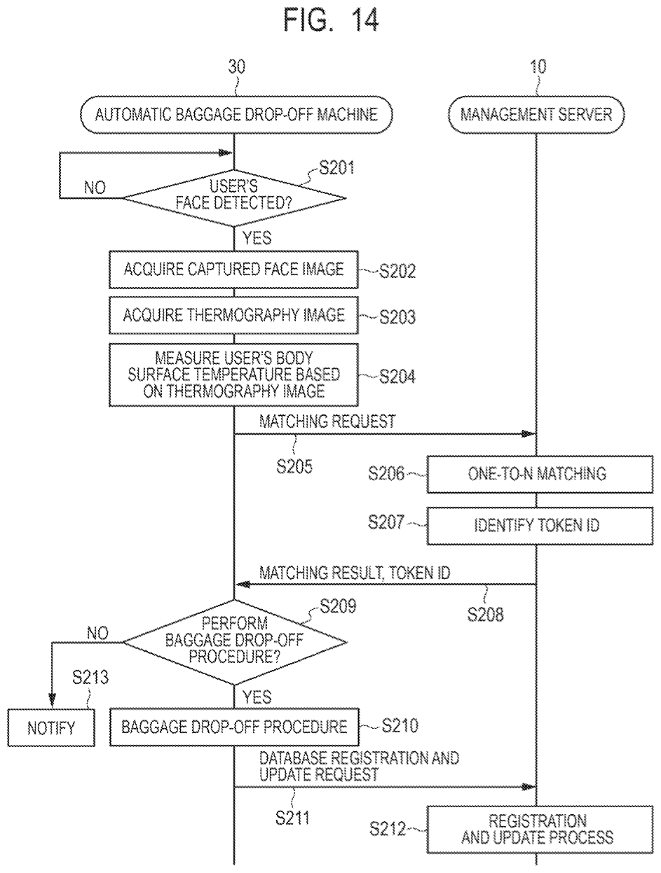

FIG. 14 is a sequence chart illustrating an example of the process in a baggage drop-off procedure of the information processing system according to the present example embodiment.

First, the automatic baggage drop-off machine 30 captures an image of the area in front of the terminal constantly or periodically and determines whether or not a face of a user U standing in front of the automatic baggage drop-off machine 30 is detected in the captured image (step S201). The automatic baggage drop-off machine 30 stands by until a face of a user U is detected in the image by the biometric information acquisition device 308 (step S201: NO).

If the automatic baggage drop-off machine 30 determines that a face of a user U is detected by the biometric information acquisition device 308 (step S201: YES), the automatic baggage drop-off machine 30 captures an image of the face of the user U and acquires the captured face image of the user U as a target face image (step S202).

Next, the automatic baggage drop-off machine 30 captures an image of the face of the user U by the thermography device 31 and acquires a thermography image (step S203). That is, the automatic baggage drop-off machine 30 captures a thermography image in synchronization with capturing of a captured face image.

Next, the automatic baggage drop-off machine 30 measures the body surface temperature of the user U based on the thermography image (step S204).

Next, the automatic baggage drop-off machine 30 requests the management server 10 to perform matching of face images (step S205). The data of the matching request includes a captured face image captured at the current place.

In response to receiving data on the matching request from the automatic baggage drop-off machine 30, the management server 10 performs one-to-N matching between the captured face image captured by the automatic baggage drop-off machine 30 and registered face images of registrants stored in the token ID information DB 10a (step S206).

Next, the management server 10 identifies the token ID of the user U provided that the matching result in step S206 is that the matching is successful (step S207).

Next, the management server 10 transmits the matching result and the token ID to the automatic baggage drop-off machine 30 (step S208). Further, to perform a baggage drop-off procedure, the management server 10 transmits operation information (for example, boarding reservation information or passport information) associated with the registered face image to the automatic baggage drop-off machine 30 together with the matching result.

Next, based on the matching result received from the management server 10, the automatic baggage drop-off machine 30 determines whether or not a baggage drop-off procedure for the user U is ready to be performed (step S209).

In this step, if the automatic baggage drop-off machine 30 determines that the matching result at the management server 10 is that the matching is unsuccessful and determines that the baggage drop-off procedure for the user U is not ready to be performed (step S209: NO), the automatic baggage drop-off machine 30 notifies the user U of an error message (step S213) and ends the process.

In contrast, if the automatic baggage drop-off machine 30 determines that the matching result at the management server 10 is that the matching is successful and determines that the automatic baggage drop-off procedure for the user U is ready to be performed (step S209: YES), the automatic baggage drop-off machine 30 performs the baggage drop-off procedure such as weighing of trustee baggage, issuance of baggage tags, transportation of trustee baggage (step S210).

Next, in response to the completion of the baggage drop-off procedure of the user U, the automatic baggage drop-off machine 30 transmits a database registration and update request to the management server 10 (step S211).

Then, in response to receiving the database registration and update request from the automatic baggage drop-off machine 30, the management server 10 performs a registration process and an update process on the passage history information DB 10b and the operation information DB 10c (step S212). Specifically, passage history information at the touch point TP2 and measurement history information on the body surface temperature of the user U at the touch point TP2 are registered to the passage history information DB 10b in association with the token ID.

### [Security Inspection Procedure]

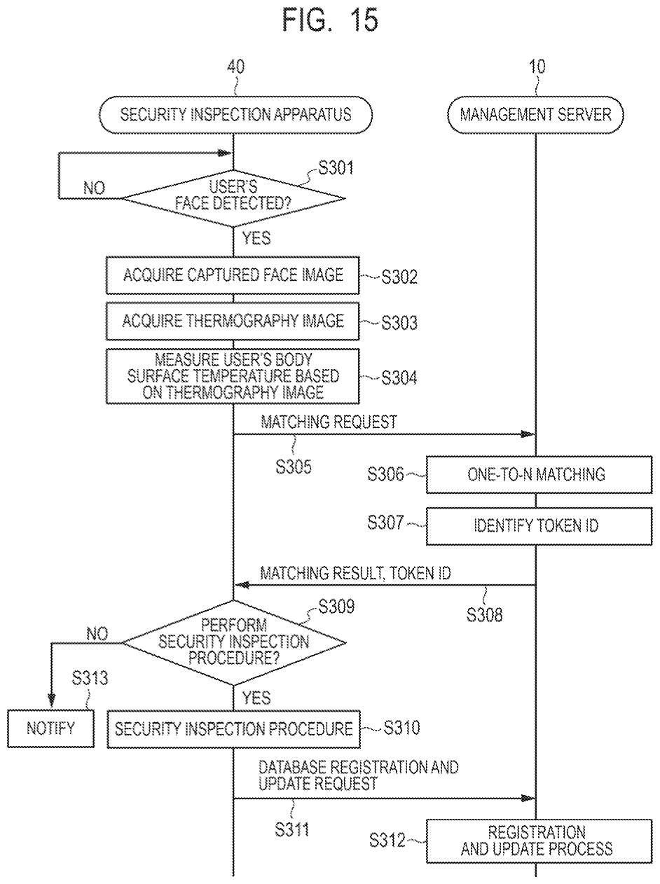

FIG. 15 is a sequence chart illustrating an example of the process in a security inspection procedure of the information processing system according to the present example embodiment.

First, the security inspection apparatus 40 captures an image of the area in front of the terminal constantly or periodically and determines whether or not a face of a user U standing in front of the security inspection apparatus 40 is detected in the captured image (step S301). The security inspection apparatus 40 stands by until a face of a user U is detected in the image by the biometric information acquisition device 408 (step S301: NO).

If the security inspection apparatus 40 determines that a face of a user U is detected by the biometric information acquisition device 408 (step S301: YES), the security inspection apparatus 40 captures an image of the face of the user U and acquires the captured face image of the user U as a target face image (step S302).

Next, the security inspection apparatus 40 captures an image of the face of the user U by the thermography device 41 and acquires a thermography image (step S303). That is, the security inspection apparatus 40 captures a thermography image in synchronization with capturing of a captured face image.

Next, the security inspection apparatus 40 measures the body surface temperature of the user U based on the thermography image (step S304).

Next, the security inspection apparatus 40 requests the management server 10 to perform matching of face images (step S305). The data of the matching request includes a captured face image captured at the current place.

In response to receiving data on the matching request from the security inspection apparatus 40, the management server 10 performs one-to-N matching between the captured face image captured by the security inspection apparatus 40 and registered face images of registrants stored in the token ID information DB 10a (step S306).

Next, the management server 10 identifies the token ID of the user U provided that the matching result in step S306 is that the matching is successful (step S307).

Next, the management server 10 transmits the matching result and the token ID to the security inspection apparatus 40 (step S308). Further, to perform a security inspection procedure, the management server 10 transmits operation information (for example, boarding reservation information or passport information) associated with the registered face image to the security inspection apparatus 40 together with the matching result.

Next, based on the matching result received from the management server 10, the security inspection apparatus 40 determines whether or not a security inspection procedure for the user U is ready to be performed (step S309).

In this step, if the security inspection apparatus 40 determines that the matching result at the management server 10 is that the matching is unsuccessful and determines that the security inspection procedure for the user U is not ready to be performed (step S309: NO), the security inspection apparatus 40 notifies the user U of an error message (step S313) and ends the process.

In contrast, if the security inspection apparatus 40 determines that the matching result at the management server 10 is that the matching is successful and determines that the security inspection procedure for the user U is ready to be performed (step S309: YES), the security inspection apparatus 40 performs the security inspection procedure such as body inspection by the metal detector and baggage inspection by X-ray machine (step S310).

Next, in response to the completion of the security inspection procedure of the user U, the security inspection apparatus 40 transmits a database registration and update request to the management server 10 (step S311).

Then, in response to receiving the database registration and update request from the security inspection apparatus 40, the management server 10 performs a registration process and an update process on the passage history information DB 10b and the operation information DB 10c (step S312). Specifically, passage history information at the touch point TP3 and measurement history information on the body surface temperature of the user U at the touch point TP3 are registered to the passage history information DB 10b in association with the token ID.

### [Departure Inspection Procedure]

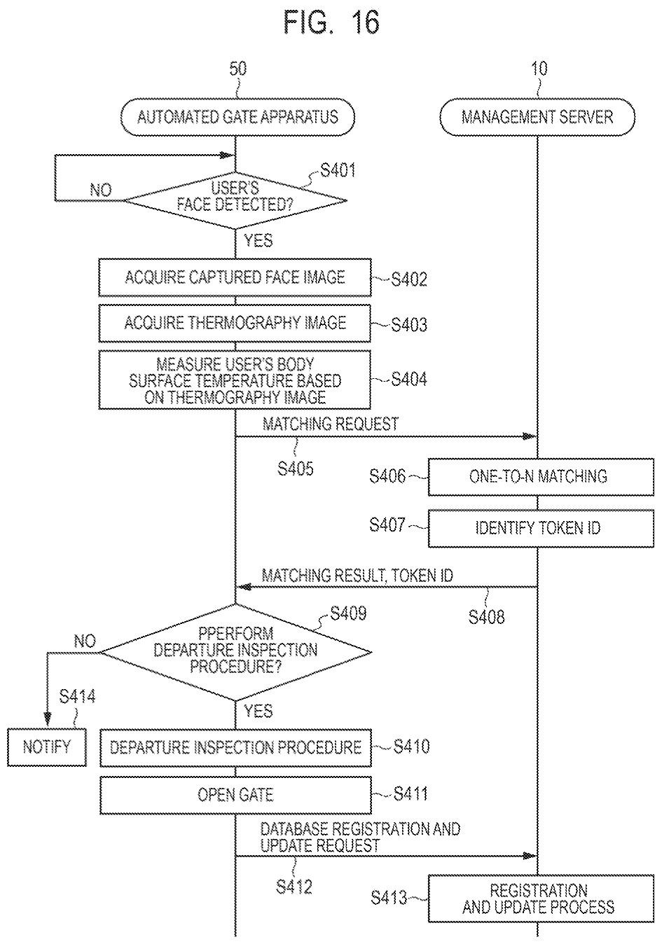

FIG. 16 is a sequence chart illustrating an example of the process in a departure inspection procedure of the information processing system according to the present example embodiment.

First, the automated gate apparatus 50 captures an image of the area in front of the terminal constantly or periodically and determines whether or not a face of a user U standing in front of the automated gate apparatus 50 is detected in the captured image (step S401). The automated gate apparatus 50 stands by until a face of a user U is detected in the image by the biometric information acquisition device 508 (step S401: NO).

If the automated gate apparatus 50 determines that a face of a user U is detected by the biometric information acquisition device 508 (step S401: YES), the automated gate apparatus 50 captures an image of the face of the user U and acquires the captured face image of the user U as a target face image (step S402).

Next, the automated gate apparatus 50 captures an image of the face of the user U by the thermography device 51 and acquires a thermography image (step S403). That is, the automated gate apparatus 50 captures a thermography image in synchronization with capturing of a captured face image.

Next, the automated gate apparatus 50 measures the body surface temperature of the user U based on the thermography image (step S404).

Next, the automated gate apparatus 50 requests the management server 10 to perform matching of face images (step S405). The data of the matching request includes a captured face image captured at the current place.

In response to receiving data on the matching request from the automated gate apparatus 50, the management server 10 performs one-to-N matching between the captured face image captured by the automated gate apparatus 50 and registered face images of registrants stored in the token ID information DB 10a (step S406).

Next, the management server 10 identifies the token ID of the user U provided that the matching result in step S406 is that the matching is successful (step S407).

Next, the management server 10 transmits the matching result and the token ID to the automated gate apparatus 50 (step S408). Further, to perform a departure inspection procedure, the management server 10 transmits operation information (for example, boarding reservation information or passport information) associated with the registered face image to the automated gate apparatus 50 together with the matching result.

Next, based on the matching result received from the management server 10, the automated gate apparatus 50 determines whether or not a departure inspection procedure for the user U is ready to be performed (step S409).

In this step, if the automated gate apparatus 50 determines that the matching result at the management server 10 is that the matching is unsuccessful and determines that the departure inspection procedure for the user U is not ready to be performed (step S409: NO), the automated gate apparatus 50 notifies the user U of an error message (step S414) and ends the process.

In contrast, if the automated gate apparatus 50 determines that the matching result at the management server 10 is that the matching is successful and determines that the departure inspection procedure for the user U is ready to be performed (step S409: YES), the automated gate apparatus 50 performs the departure inspection procedure such as body inspection by the metal detector and baggage inspection by X-ray machine (step S410).

Next, if the user U is allowed to departure by the departure inspection procedure, the automated gate apparatus 50 opens the gate 510 (step S411).

Next, in response to the completion of the departure inspection procedure of the user U, the automated gate apparatus 50 transmits a database registration and update request to the management server 10 (step S412).

Then, in response to receiving the database registration and update request from the automated gate apparatus 50, the management server 10 performs a registration process and an update process on the passage history information DB 10b and the operation information DB 10c (step S413). Specifically, passage history information at the touch point TP4 and measurement history information on the body surface temperature of the user U at the touch point TP4 are registered to the passage history information DB 10b in association with the token ID.

### [Identity Verification Procedure at Boarding Gate]

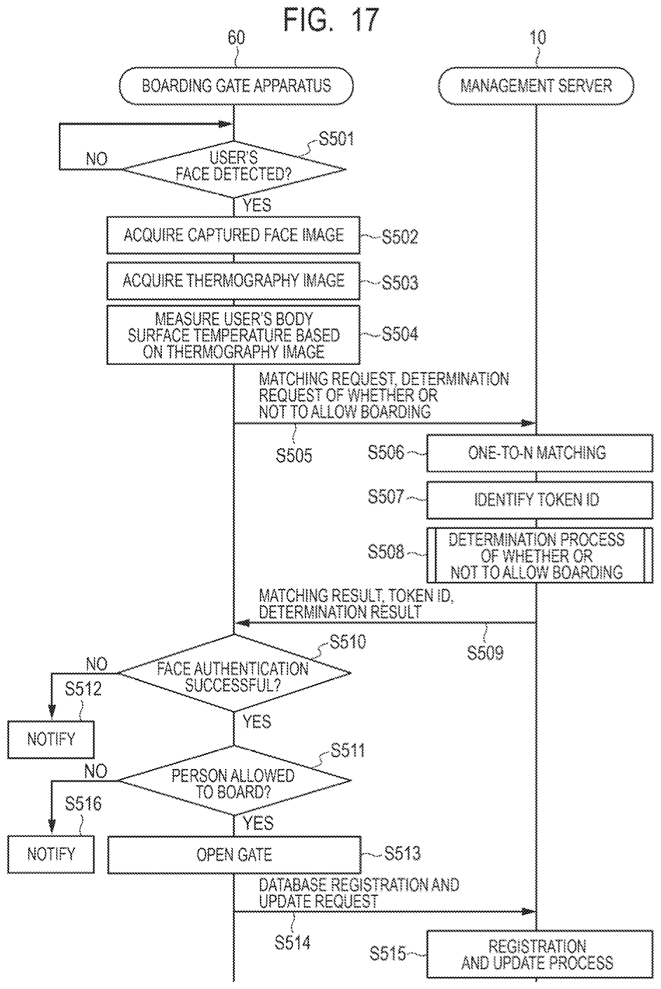

FIG. 17 is a sequence chart illustrating an example of the process in an identity verification procedure at the boarding gate of the information processing system according to the present example embodiment.

First, the boarding gate apparatus 60 captures an image of the area in front of the terminal constantly or periodically and determines whether or not a face of a user U standing in front of the boarding gate apparatus 60 is detected in the captured image (step S501). The boarding gate apparatus 60 stands by until a face of a user U is detected in the image by the biometric information acquisition device 608 (step S501: NO).

If the boarding gate apparatus 60 determines that a face of a user U is detected by the biometric information acquisition device 608 (step S501: YES), the boarding gate apparatus 60 captures an image of the face of the user U and acquires the captured face image of the user U as a target face image (step S502).

Next, the boarding gate apparatus 60 captures an image of the face of the user U by the thermography device 61 and acquires a thermography image (step S503). That is, the boarding gate apparatus 60 captures a thermography image in synchronization with capturing of a captured face image.

Next, the boarding gate apparatus 60 measures the body surface temperature of the user U based on the thermography image (step S504).

Next, the boarding gate apparatus 60 requests the management server 10 to perform the matching process of face images and the determination process of whether or not to allow boarding (step S505). The data of the matching request includes a captured face image captured at the current place.

In response to receiving data on the matching request from the boarding gate apparatus 60, the management server 10 performs one-to-N matching between the captured face image captured by the boarding gate apparatus 60 and registered face images of registrants stored in the token ID information DB 10a (step S506).

Next, the management server 10 identifies the token ID of the user U provided that the matching result in step S506 is that the matching is successful (step S507).

Next, using the token ID of the user U as a key, the management server 10 refers to the measurement history information of the body surface temperature of the user U stored in the passage history information DB 10b, and performs determination process of whether the user U is allowed to board the airplane (step S508). Details of the determination process of whether or not the user is allowed to board the airplane will be described later.

Next, the management server 10 transmits the matching result, the token ID, and determination result of whether or not to allow boarding to the boarding gate apparatus 60 (step S509). Further, to perform the identity verification procedure at the boarding gate, the management server 10 transmits operation information (for example, boarding reservation information or passport information) associated with the registered face image to the boarding gate apparatus 60 together with the matching result.

Next, the boarding gate apparatus 60 determines whether or not face authentication of the user U is successful at the management server 10 (step S510).

In this step, if the boarding gate apparatus 60 determines that the matching result at the management server 10 is that the matching is unsuccessful and determines that the face authentication of the user U failed (step S510: NO), the boarding gate apparatus 60 notifies the user U of an error message (step S512) and ends the process.

In contrast, if the boarding gate apparatus 60 determines that the matching result at the management server 10 is that the matching is successful and determines that the face authentication of the user U is successful (step S510: YES), the process proceeds to step S511.

In step S511, the boarding gate apparatus 60 refers to the determination result at the management server 10 and determines whether or not the user U is the person allowed to board the airplane.

In this step, if the boarding gate apparatus 60 determines that the user U is not the person allowed to board the airplane (step S511: NO), the boarding gate apparatus 60 notifies the user U of an error message (step S516) and ends the process.

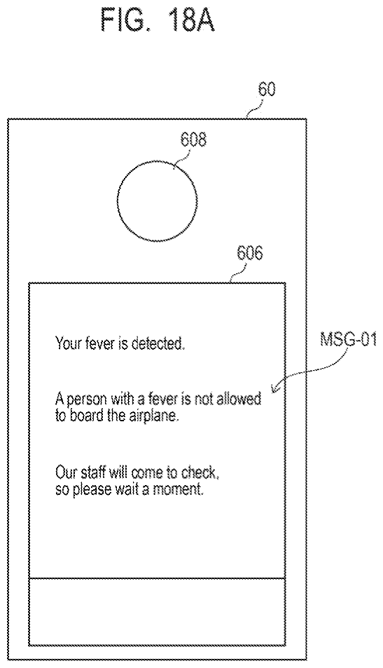

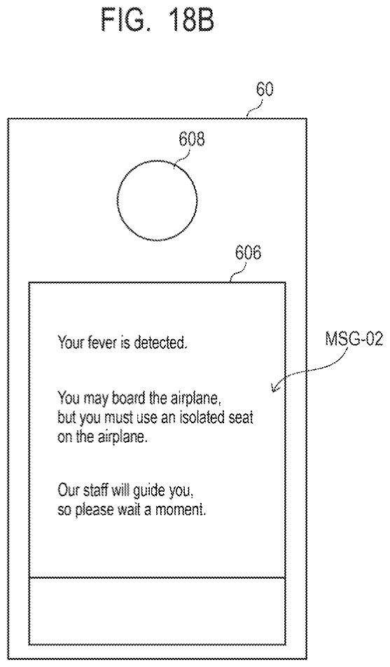

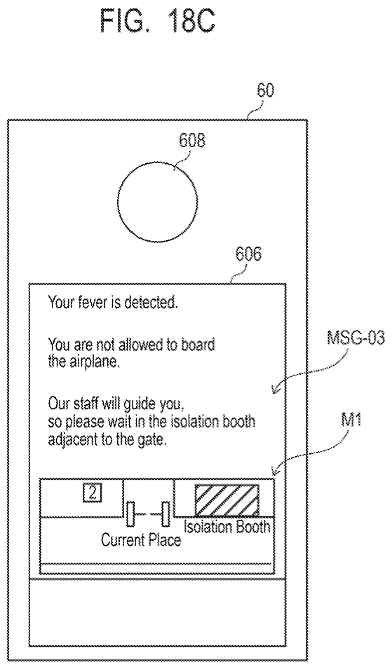

FIGS. 18A-18C are a diagram illustrating an example of a screen displayed on the boarding gate apparatus 60. In FIG. 18A, an error message MSG-01 is displayed on the display device 606 of the boarding gate apparatus 60 to refuse boarding to the user U. The error message MSG-01 is as follows. “Your fever state is detected. A person with a fever is not allowed to board the airplane. Our staff will come to check, so please wait a moment.”

In step S511 described above, the boarding of User U was determined in two categories of “allowance” or “refusal”, but it may be determined by adding a category of “Marked (Observation required)”. In FIG. 18B, a message MSG-02 is displayed to inform the user U of the state of fever and seat change. The message MSG-02 is as follows. “Your fever state is detected. You may board the airplane, but you must use an isolated seat on the airplane. Our staff will guide you, so please wait a moment.” In other words, the user U determined to be “Marked” is allowed to board the airplane, but is required to sit in a dedicated seat in an isolation zone for marked persons on board the airplane. In this way, the marked persons are isolated from other passengers and gathered in one place on the airplane, so that they can be conditionally allowed to travel by the user U while controlling the spread of infectious diseases on board the airplane.

In FIG. 18B, the boarding gate apparatus 60 may guide the user U to the changed seat and the moving route to the seat, etc. Specifically, the boarding gate apparatus 60 may display the changed seat position and seat number on the screen. Similarly, the boarding gate apparatus 60 may display a two-dimensional bar code including the seat position and seat number on the screen. Furthermore, the boarding gate apparatus 60 may print information such as the two-dimensional bar code, seat position, and seat number on a recording medium and provide it to the user U.

In addition, in step S516, it is possible to show the place of the isolation booth or the inspection booth as well as the error message of denial of boarding. In FIG. 18C, a message MSG-03 to guide the user U to the place of the isolation booth and a guide map M1 to guide the route from the current place to the isolation booth are displayed. The message MSG-03 is as follows. “Your fever state is detected. You are not allowed to board the airplane. Our staff will guide you, so please wait in the isolation booth adjacent to the gate.”

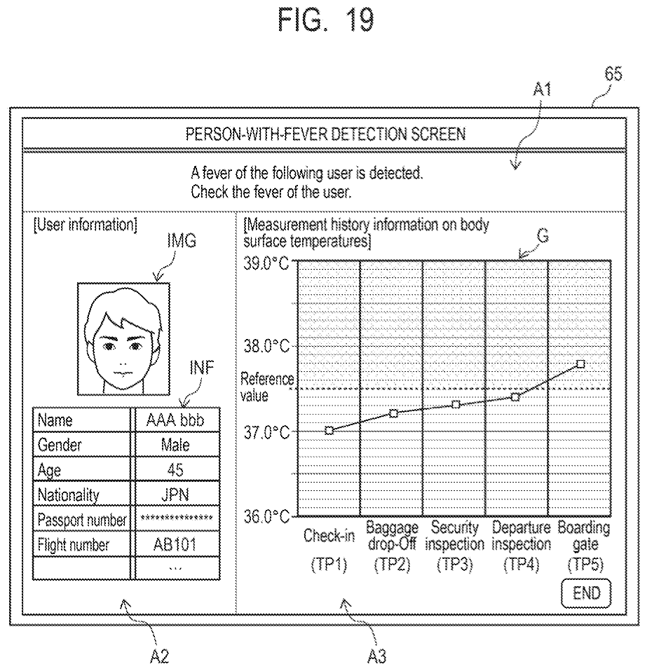

FIG. 19 is a diagram illustrating an example of a screen displayed on the operation terminal 65. In the upper column A1 of the screen, an error message indicating that the user U was denied boarding the airplane is displayed. The error message is as follows. “A fever state in the following user is detected. Check the fever state of the user.” In the lower left column A2 of the screen, the user U's face image IMG and user information INF are displayed. In the lower right column A3 of the screen, graph G of the measurement history information of the body surface temperature measured at each touch points TP1 to TP5 of the user U is displayed. According to the graph G, it can be seen that the body surface temperature of the user U is below the reference value (37.5 degrees Celsius) from the check-in procedure to the departure inspection procedure, but exceeds the reference value at the boarding gate.

In contrast, the boarding gate apparatus 60 determines that the user U is the person allowed to board the airplane (step S511: YES), the process proceeds to step S513.

In step S513, the boarding gate apparatus 60 opens the gate 610. Accordingly, the user U passes through the boarding gate apparatus 60 and boards the airplane.

Next, in response to the completion of the identity verification of the user U and completion of determination process of whether or not to allow boarding, the boarding gate apparatus 60 transmits a database registration and update request to the management server 10 (step S514).

Then, in response to receiving the database registration and update request from the boarding gate apparatus 60, the management server 10 performs a registration process and an update process on the passage history information DB 10b and the operation information DB 10c (step S515). Specifically, passage history information at the touch point TP5 and measurement history information on the body surface temperature of the user U at the touch point TP5 are registered to the passage history information DB 10b in association with the token ID.

### [Determination Process of Whether or not to Allow Boarding]

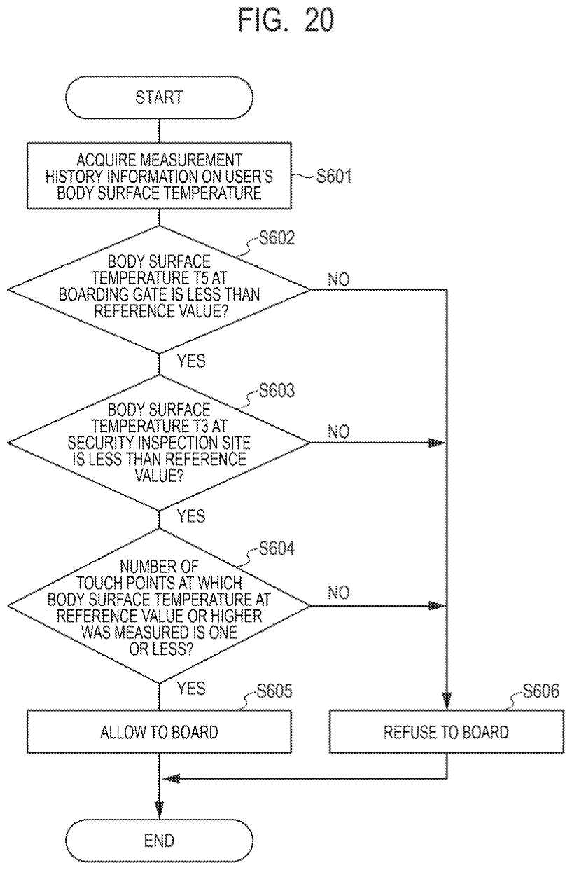

FIG. 20 is a flowchart illustrating an example of a process for determining whether or not to allow boarding in the management server 10 according to the present example embodiment.

First, if the management server 10 refers to the passage history information DB 10b with the token ID identified for the user U as a key, the management server 10 acquires the measurement history information of the body surface temperature of the user U (step S601). Specifically, the management server 10 acquires the body surface temperatures T1 to T5 measured for the user U at the touch points TP1 to TP5, respectively.

Next, the management server 10 determines whether or not the body surface temperature T5 at the boarding gate (touch point TP5) is less than a predetermined reference value (step S602). In the present example embodiment, the reference value is described as 37.5 degrees Celsius.

In this step, if the management server 10 determines that the body surface temperature T5 at the boarding gate is less than a predetermined reference value (step S602: YES), the process proceeds to step S603.

In contrast, if the management server 10 determines that the body surface temperature T5 at the boarding gate is higher than or equal to a predetermined reference value (step S602: NO), the process proceeds to step S606.

In step S603, the management server 10 determines whether or not the body surface temperature T3 at the security inspection site (touch point TP3) is less than the prescribed reference value.

If the management server 10 determines that the body surface temperature T3 at the security inspection site is less than the predetermined reference value (step S603: YES), the process proceeds to step S604.

In contrast, if the management server 10 determines that the body surface temperature T3 at the security inspection site is higher than or equal to the predetermined reference value (step S603: NO), the process proceeds to step S606.

In step S604, the management server 10 determines whether the total number of touch points where a body surface temperature is higher than or equal to the reference value is measured out of the touch points TP1 to TP5 is one or less.

If the management server 10 determines that the total number of touch points where the body surface temperature higher than or equal to the reference value is measured out is one or less (step S604: YES), the process proceeds to step S605.

In contrast, if the management server 10 determines that the total number of touch points where the body surface temperature is higher than or equal to the reference value is two or greater (step S604: NO), the process proceeds to step S606. The total number of touch points, which is the criterion in step S604, is not limited to one. For example, the total number may be zero instead of one.

In step S605, the management server 10 outputs a determination result indicating that the user U is allowed to board the airplane.

In contrast, in step S606, the management server 10 outputs a determination result indicating that the user U is refused to board the airplane.

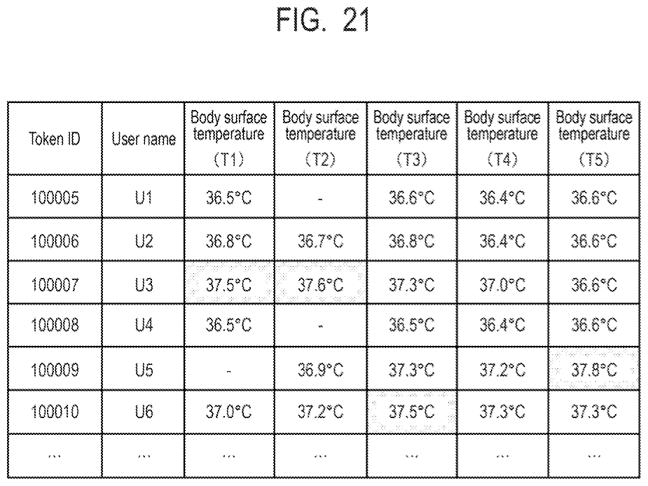

FIG. 21 is a diagram illustrating an example of measurement history information on body surface temperatures for each users U measured at each touch point. The body surface temperatures T1 to T5 are the body surface temperatures measured at the check-in counter (touch point TP1), the baggage counter (touch point TP2), the security inspection site (touch point TP3), the departure inspection site (touch point TP4) and the boarding gate (touch point TP5), respectively.

For user U1 having a token ID of “100005”, four values of body surface temperature T1 and T3 to T5 are acquired, and all values are lower than the reference value (37.5 degrees Celsius). In such a case, the management server 10 outputs the determination result of allowance of boarding for the user U1.

For user U2 having a token ID of “100006”, five values of body surface temperature T1 to T5 are acquired, and all values are lower than the reference value (37.5 degrees Celsius). In such a case, the management server 10 outputs the determination result of allowance of boarding for the user U2.

For user U3 having a token ID of “100007”, five values of body surface temperature T1 to T5 are acquired. In these values, two values of body surface temperature T1 and body surface temperature T2 are higher than or equal to the reference value (37.5 degrees Celsius). In such a way, if the body surface temperature T5 at the boarding gate is lower than the reference value, but a body surface temperature higher than or equal the reference value is recorded at two or more touch points (hereafter referred to as “boarding refusal pattern A”), the management server 10 outputs the determination result of refusal of boarding.

According to the boarding refusal pattern A, even if, for example, the user U repeats a fever state and a non-fever state at the airport DA, the user U can be detected as a person with a fever. The boarding refusal pattern A corresponds to the case where “NO” is determined in step S604 of FIG. 20.

For the user U4 having a token ID of “100008”, four values of body surface temperature T1 and T2 to T5 are acquired, and all values are lower than the reference value (37.5 degrees Celsius). In such a case, the management server 10 outputs the determination result of allowance of boarding to the user U4.

For the user U5 having a token ID of “100009”, four values of body surface temperature T2 to T5 are acquired. In these values, the value of body surface temperature T5 is higher than or equal to the reference value (37.5 degrees Celsius). In such a way, if all of the body surface temperatures measured at touch points other than the boarding gate are lower than the reference value, the body surface temperature T5 at the last touch point TP5 (boarding gate) is higher than or equal to the reference value (hereafter referred to as “boarding refusal pattern B”), the management server 10 outputs the determination result of refusal of boarding.

According to the boarding refusal pattern B, it is possible to determine whether or not to board with an emphasis on a body surface temperature in a procedure immediately before boarding among a plurality of body surface temperatures at a plurality of touch points. The boarding refusal pattern B corresponds to the case where “NO” is determined in step S602 of FIG. 20.

For the user U6 having a token ID of “100010”, five values of body surface temperature T1 to T5 are acquired. In these values, only the value of body surface temperature T3 is higher than or equal to the reference value (37.5 degrees Celsius). In such a way, if all the body surface temperatures measured at the touch points other than the touch point TP3 (security inspection area) are lower than the reference value, the body surface temperature T3 at the touch point TP3 through which all the users U pass is higher than or equal to the reference value (hereafter referred to as “boarding refusal pattern C”), the management server 10 outputs the determination result of the refusal of boarding.

According to the boarding refusal pattern C, it is possible to to determine whether or not to board with an emphasis on a body surface temperature at a particular touch point among a plurality of body surface temperatures at a plurality of touch points. The boarding refusal pattern C corresponds to the case where “NO” is determined in step S603 of FIG. 20.

Note that the boarding refusal pattern is not limited to the three boarding refusal patterns A-C described above and can be set as any pattern. The order of processes of the boarding refusal patterns A-C is not limited to the order illustrated in FIG. 20. For example, the order of steps S602, S603, and S604 in FIG. 20 may be changed to the order of steps S604, S602, and S603.

As described above, the management server 10 according to the present example embodiment determines whether or not the user U is allowed to board an airplane based on a plurality of body surface temperatures measured for the same user U at a plurality of touch points TP1 to TP5 in the airport DA.

In detail, the determination is not based solely on the body surface temperature measured at a single touch point, but based on measurement history information including a plurality of body surface temperatures measured at a plurality of touch points at different times, the infection risk of the user U is determined comprehensively. This enables the management server 10 to reliably detect the user U as a person with a health problem, that is, a person at risk of infection, even if, for example, the user U who was in a fever state at the time of check-in has subsided during the procedure at the boarding gate. As a result, it can contribute to preventing the spread of infection in the airport and the airplane.

### Second Example Embodiment

The information processing system in the present example embodiment will be described below. Note that references common to the references provided in the drawings in the first example embodiment represent the same components. Description of the features common to the first example embodiment will be omitted, and different features will be described in detail.

The present example embodiment differs from the first example embodiment in that the determination process of whether or not to board for the user U is performed not only at the last touch point TP5 (boarding gate) of the airport DA but also at the touch point TP3 (security inspection site) through which all the users U need to pass.

### [Security Inspection Procedure]

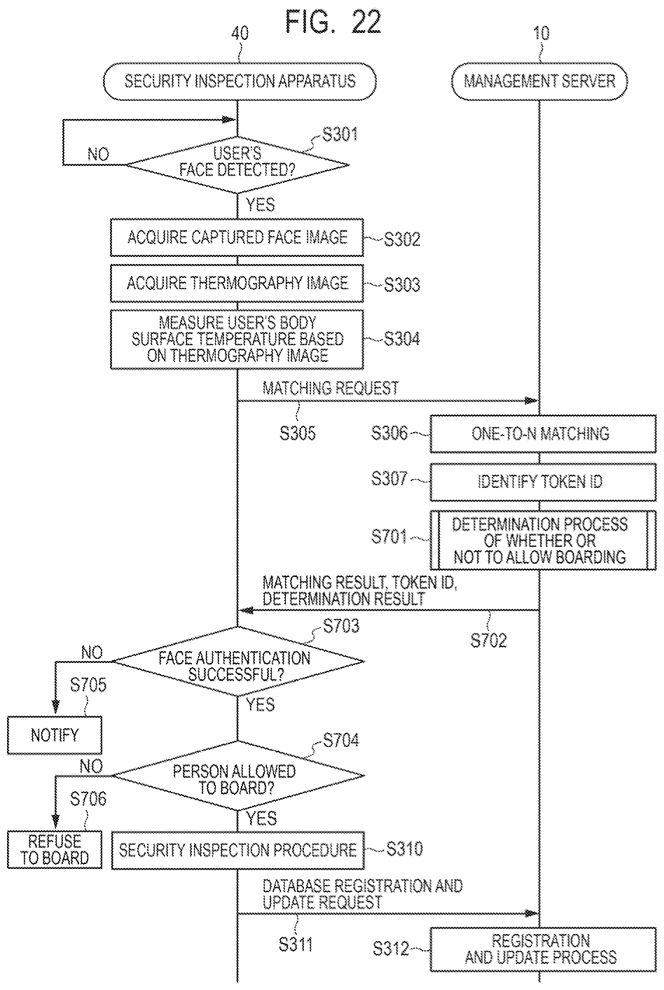

FIG. 22 is a sequence chart illustrating an example of a process in a security inspection procedure of the information processing system according to the present example embodiment. The process of FIG. 22 is similar to the process of FIG. 15 described above, except for steps S701 to S706. Therefore, the differences are described below.

In step S307, if the management server 10 identifies the token ID of the user U on the condition that the matching result in step S306 is that the matching is successful, the process proceeds to step S701.

In step S701, the management server 10 uses the token ID of the user U as a key to refer to the body surface temperature of the user U stored in the passage history information DB 10b, and performs the determination process to determine whether or not the user U is allowed to board the airplane. Details of the determination process to determine whether or not the user is allowed to board the airplane will be described later.

Next, the management server 10 transmits the matching result, the token ID, and the determination result of whether or not the user is allowed to board the airplane to the security inspection apparatus 40 (step S702). The management server 10 also transmits the operation information (For example, boarding reservations and passport information) associated with the registered face image to the security inspection apparatus 40 together with the matching result for the execution of the security inspection procedure.

Next, the security inspection apparatus 40 determines whether or not the face authentication of the user U is successful in the management server 10 (step S703).

In this step, if the security inspection apparatus 40 determines that the matching result in the management server 10 is unsuccessful and determines that the face authentication of the user U is unsuccessful (step S703: NO), the security inspection apparatus 40 notifies the user U of an error message (step S705) and ends the process.

In contrast, if the security inspection apparatus 40 determines that the matching result in the management server 10 is successful and determines that the face authentication of the user U is successful (step S703: YES), the process proceeds to step S704.

In step S704, the security inspection apparatus 40 refers to the determination result in the management server 10 and determines whether or not the user U is a person allowed to board an airplane.

In this step, if the security inspection apparatus 40 determines that the user U is not a person allowed to board the airplane (step S704: NO), the security inspection apparatus 40 notifies the user U of an error message of boarding refusal (step S706) and ends the process.

In contrast, if the security inspection apparatus 40 determines that the user U is a person allowed to board an airplane (step S706: YES), the process proceeds to step S310. The process from step S310 onward is the same as that in FIG. 15 described above.

### [Determination Process of Whether or not to Allow Boarding (2)]

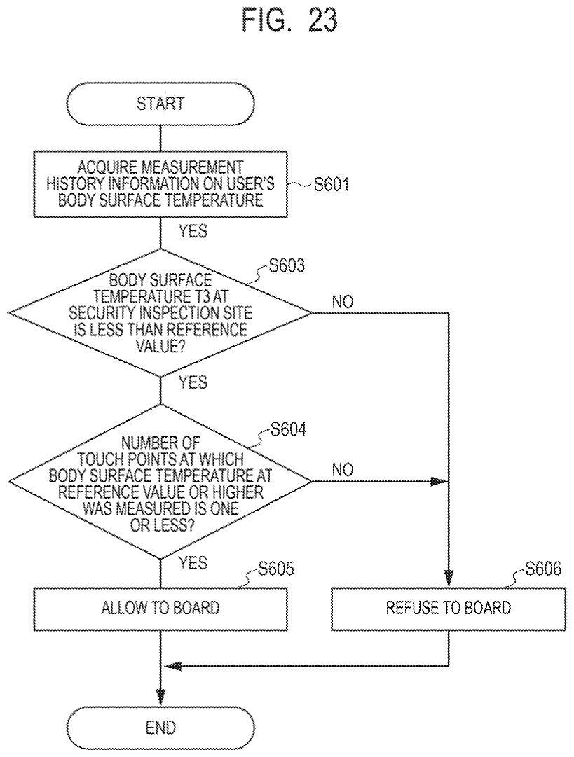

FIG. 23 is a flowchart illustrating an example of a process for determining whether or not to allow boarding in the management server 10 according to the present example embodiment. In the process of FIG. 23, step S602 is omitted from the above FIG. 20. Therefore, the difference in the process content from that of FIG. 20 will be described.

In step S601, the management server 10 refers to the passage history information DB 10b with the token ID identified for the user U as a key, and acquires the measurement history information of the body surface temperature of the user U.

Specifically, body surface temperatures T1-T3 measured for the user U from the touch point TP1 (check-in counter) to the touch point TP3 (security inspection site) are acquired, respectively. Then, the process proceeds to step S603. The process of step S603 is the same as that in FIG. 20.

In step S604, the management server 10 determines whether the total number of touch points where a body surface temperature is higher than or equal to the reference value is measured out of the touch points TP1 to TP3 is one or less.

If the management server 10 determines that the total number of touch points where the body surface temperature higher than or equal to the reference value is measured out is one or less (step S604: YES), the process proceeds to step S605.

In contrast, if the management server 10 determines that the total number of touch points where the body surface temperature is higher than or equal to the reference value is two or larger (step S604: NO), the process proceeds to step S606. The processes of steps S605 and S606 are the same as in FIG. 20.

As described above, the management server 10 according to the present example embodiment can execute the determination process of whether or not the user U is allowed to board the airplane at the intermediate touch point TP3 (security inspection site) through which all the users U must pass. Thus, in addition to the effect of the first example embodiment, it has the effect of early detection of the user U having an infection risk.

### Third Example Embodiment

The information processing system in the present example embodiment will be described below. Note that references common to the references provided in the drawings in the first example embodiment represent the same components. Description of the features common to the first example embodiment will be omitted, and different features will be described in detail.

The present example embodiment differs from the first example embodiment in that it further has a function to estimate the basal body temperature of the user U, that is, normal body temperature (Hereafter referred to as “normal temperature”.), based on the body temperature history information registered in advance from the user U to the information processing system, and to change the reference value for determination in the determination process of boarding propriety based on this normal body temperature.

### [Determination Process of Whether or not to Allow Boarding (3)]

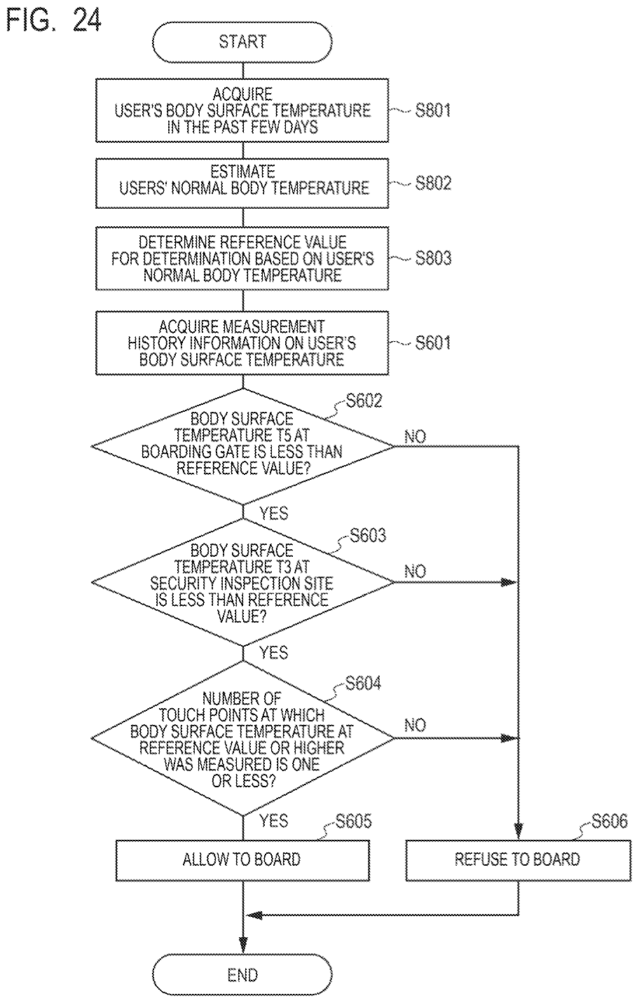

FIG. 24 is a flowchart illustrating an example of the decision process of boarding propriety in the management server 10 according to the present example embodiment. The process in FIG. 24 is the same as the process in FIG. 20 described above, except for steps S801 to S803. Therefore, the differences are described below.

In step S801, the management server 10 acquires the temperature history information of the user U in the past several days with the departure date as the reference date. The temperature history information is, for example, registered by the user U in the database (not illustrated) of the management server 10 or in the airline reservation system (not illustrated) via the network. It is also preferable that a predetermined application is installed in advance in a portable terminal such as a smartphone owned by the user U and the temperature history information can be registered via the application.

In step S802, the management server 10 estimates the normal temperature of the user U based on the temperature history information of the user U. For example, the management server 10 estimates the average value of the body temperature for one week as the normal temperature.

In step S803, if the management server 10 determines or changes the reference value for determination based on the normal temperature, the process proceeds to step S601. As an example, in the case of the user U whose normal temperature is lower than a predetermined value (e.g., 36.5 degrees Celsius), the reference value is made lower than the initial value (e.g., 37.5 degrees Celsius). Conversely, in the case of the user U whose normal temperature is higher than a predetermined value, the reference value is made higher than the initial value. However, in the case where the normal temperature is high and it is difficult to distinguish the user U from the fever state, the reference value need not be changed.

As described above, according to the present example embodiment, in addition to the effect of the first example embodiment, by considering the normal temperature for each user U, the effect that the determination accuracy of whether or not the user U is allowed to board can be further improved can be achieved.

### Fourth Example Embodiment

The information processing system in the present example embodiment will be described below. Note that references common to the references provided in the drawings in the first example embodiment represent the same components. Description of the features common to the first example embodiment will be omitted, and different features will be described in detail.

The present example embodiment differs from the first example embodiment in that the reference value for determination in the determination process of whether or not to allow boarding is changed based on the attribute information of the user U obtained by face authentication. The attribute information includes, but is not limited to, age, gender, nationality, etc. of the user U, for example. It is sufficient if the attribute information is medically or statistically correlated with the value of normal temperature.

### [Determination Process of Whether or not to Allow Boarding (4)]

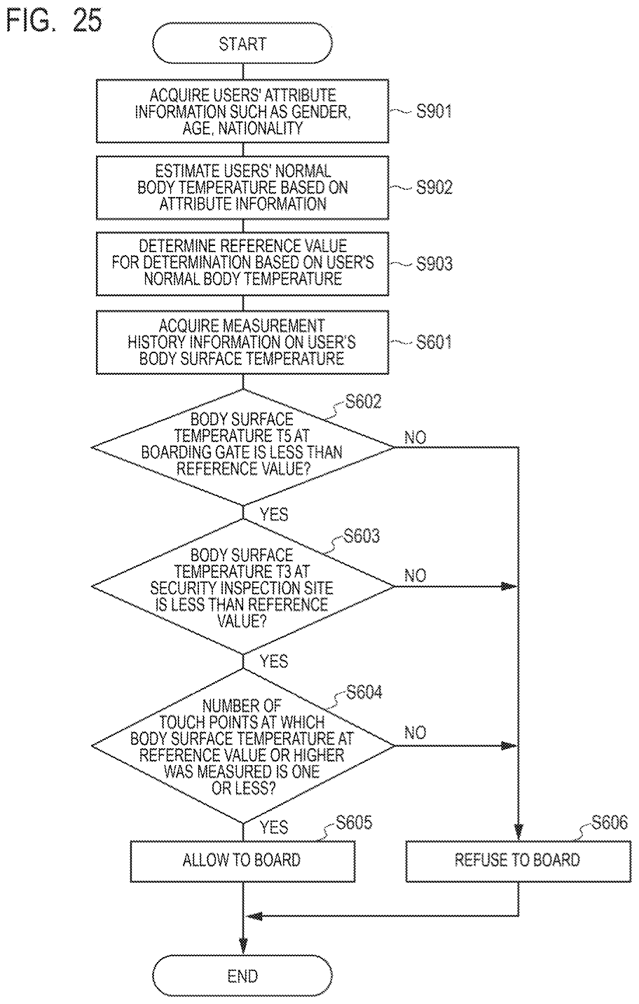

FIG. 25 is a flowchart illustrating an example of the determination process for boarding permission in the management server 10 according to the present example embodiment. The process of FIG. 25 is the same as the process of FIG. 20 described above, except for steps S901 to S903. Therefore, the differences are described below.

In step S901, the management server 10 refers to the operation information DB 10c based on the token ID of the identified user U, and acquires attribute information such as gender, age, and nationality of the user U.

In step S902, the management server 10 estimates the normal temperature of the user U based on the attribute information of the user U. For example, the management server 10 estimates the normal temperature according to the age of the user U. The number of attribute information items used in estimating the normal temperature is not limited to one item, and a plurality of items may be combined.

In step S803, if the management server 10 changes the reference value for determination based on the normal temperature, the process proceeds to step S601. For example, the normal temperature of infants and children tends to be higher than that of adults. Also, among adults, the normal temperature of the elderly tends to be relatively low. Therefore, if using age in attribute information, it is preferable to change the reference value for determination according to the age group. The same applies when there is a difference in the value of normal temperature depending on the nationality or gender of the user U.

As described above, according to the present example embodiment, in addition to the effect of the first example embodiment, by considering the attribute information for each user U, an effect that can further improve the determination accuracy of whether or not to allow boarding for the user U.

### Fifth Example Embodiment

The information processing system in the present example embodiment will be described below. Note that references common to the references provided in the drawings in the first example embodiment represent the same components. Description of the features common to the first example embodiment will be omitted, and different features will be described in detail.

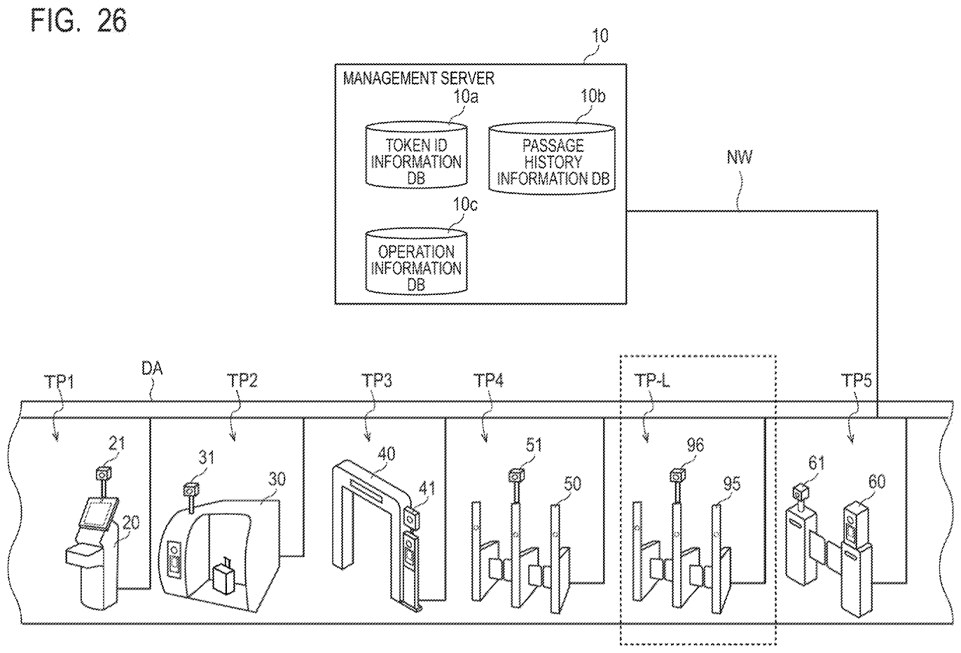

FIG. 26 is a block diagram illustrating an example of the overall configuration of the information processing system according to the present example embodiment. In this step, a case is illustrated in which the touch point TP-L is separately provided between the touch point TP4 (departure inspection site) and the touch point TP5 (boarding gate). The touch point TP-L is, for example, a lounge. The touch point TP-L is equipped with an automated gate apparatus 95. The configuration of the automated gate apparatus 95 is similar to that of the automated gate apparatus 50 described above and is equipped with thermography 96.

If the user U enters or exits the lounge (touch point TP-L), the automated gate apparatus 95 captures a face image and a thermographic image of the user U, and the automated gate apparatus 95 transmits data to the management server 10. Thus, as in the case of other touch points TP1 to TP5, the management server 10 can register the history information of the body surface temperature of the user U in the passage history information DB 10b. When the user U performs the procedure at the boarding gate (touch point TP5), the management server 10 determines whether or not the user U can board by considering the body surface temperature of the user U measured at the touch point TP-L in addition to the body surface temperatures measured at the touch points TP1 to TP5.

As described above, according to the present example embodiment, by considering the body surface temperature measured at a location in the airport DA other than those locations where the procedure for departure is performed (touch points TP1 to TP5), the determination accuracy of whether or not the user U is allowed to board can be further improved.

### Sixth Example Embodiment

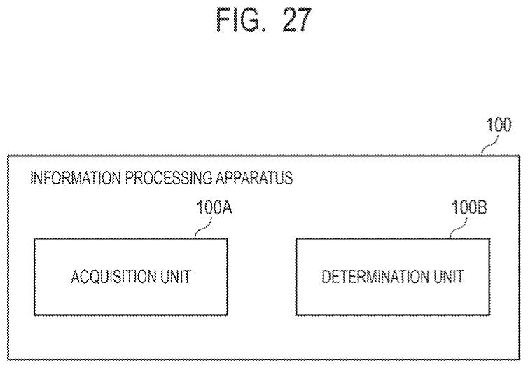

FIG. 27 is a function block diagram of an information processing apparatus 100 according to the present example embodiment. The information processing apparatus 100 includes an acquisition unit 100A and a determination unit 100B. The acquisition unit 100A acquires body surface temperatures of a user of an airplane measured at procedure places of a plurality of procedures imposed on the user at a departure airport. The determination unit 100B determines whether the user is allowed to board the airplane based on the plurality of body surface temperatures acquired from the user.

According to the present example embodiment, the information processing apparatus 100 that can efficiently detect a person with a health problem at a departure airport is provided.

### [Modified Example Embodiment]

Although the present invention has been described above with reference to the example embodiments, the present invention is not limited to the example embodiments described above. Various modifications that can be understood by those skilled in the art can be made to the configuration and details of the present invention within the scope not departing from the spirit of the present invention. For example, it should be understood that an example embodiment in which a configuration of a part of any of the example embodiments is added to another example embodiment or an example embodiment in which a configuration of a part of any of the example embodiments is replaced with a configuration of a part of another example embodiment is also an example embodiment to which the present invention may be applied.

For example, a weighting value may be preset for each of the plurality of touch points TP1 to TP5. In this case, the management server 10 can determine whether or not the user U can board the airplane based on the total score obtained by multiplying the result of the comparison processing between the body surface temperature measured at each touch point and the reference value by the weighted value of each touch point. For example, when the weighted values of the touch point TP1 to TP5 are set to gradually increase as the touch point approaches the touch point TP5, the body surface temperature of the user U measured at the time closer to the scheduled departure time can be emphasized. It is preferable that the weighted values for each touch point can be changed arbitrarily.

In addition, in the above example embodiment, the determination process of whether or not the user U boarding the airplane for an international flight is allowed to board the airplane, but the system configuration according to the present invention can be similarly applied to a domestic flight.

In the above example embodiment, the determination process for comparing a plurality of body surface temperatures measured at each touch point with a predetermined reference value for a user U is described, but the management server 10 may calculate the mean value or deviation value of a plurality of body surface temperatures and compare these calculated values with the reference value. Similarly, the management server 10 may identify the maximum value among a plurality of body surface temperatures measured from the same user U and compare the maximum value with the reference value.

Further, instead of comparing the body surface temperature with the reference value, the management server 10 may calculate a variation range in a time series of a plurality of body surface temperatures measured from the user U and be configured to refuse boarding of the airplane to the user U whose variation range is higher than or equal to a predetermined value. That is, the method for determining whether or not the user U is allowed to board the airplane based on a plurality of body surface temperatures can be changed freely.

The scope of each of the example embodiments also includes a processing method that stores, in a storage medium, a program that causes the configuration of each of the example embodiments to operate so as to implement the function of each of the example embodiments described above, reads the program stored in the storage medium as a code, and executes the program in a computer. That is, the scope of each of the example embodiments also includes a computer readable storage medium. Further, each of the example embodiments includes not only the storage medium in which the program described above is stored but also the individual program itself.

As the storage medium, for example, a floppy (registered trademark) disk, a hard disk, an optical disk, a magneto-optical disk, a CD-ROM, a magnetic tape, a nonvolatile memory card, or the like can be used. Further, the scope of each of the example embodiments also includes an example that operates on OS to perform a process in cooperation with another software or a function of an add-in board without being limited to an example that performs a process by an individual program stored in the storage medium.

The whole or part of the example embodiments disclosed above can be described as, but not limited to, the following supplementary notes.

### (Supplementary Note 1)

An information processing apparatus comprising:
an acquisition unit that acquires body surface temperatures of a user of an airplane measured at procedure places of a plurality of procedures imposed on the user at a departure airport; and
a determination unit that determines whether the user is allowed to board the airplane based on the plurality of body surface temperatures acquired from the user.

### (Supplementary Note 2)

The information processing apparatus according to supplementary note 1, further comprising:
a processing unit that associates the registered face image of the user and the body surface temperature acquired at a procedure place in a check-in procedure with identification information that uniquely identifies the user; and
a face matching unit that performs face matching between the registered face image and the user's face image acquired at the location of procedure whose order is later than the check-in procedure,
wherein the processing unit associates the body surface temperature at the procedure place to the identification information when the user is authenticated by the face matching.

### (Supplementary Note 3)

The information processing apparatus according to supplementary note 1 or 2, wherein the determination unit determines whether or not the user is allowed to board the airplane based on a result of comparison between the plurality of body surface temperatures and a predetermined reference value.

### (Supplementary Note 4)

The information processing apparatus according to supplementary note 3, wherein when there are a predetermined number of values is equal to or larger than the reference value among the plurality of the body surface temperatures, the determination unit refuses the user to board the airplane.

### (Supplementary Note 5)

The information processing apparatus according to supplementary note 4, wherein the determination unit counts the number of times when the body surface temperature of the user is equal to or higher than the reference value, and refuses the user to board the airplane at the procedure place where the number of times has reached the predetermined number.

### (Supplementary Note 6)

The information processing apparatus according to any one of supplementary notes 3 to 5, wherein the determination unit refuses the user to board the airplane when a body surface temperature at a specific procedure place is equal to or higher than the reference value.

### (Supplementary Note 7)

The information processing apparatus according to any one of supplementary notes 3 to 6, wherein the determination unit changes the reference value for each user based on the basal body temperature registered in advance for each user.

### (Supplementary Note 8)

The information processing apparatus according to any one of supplementary notes 3 to 7, wherein the determination unit changes the reference value for each user based on the user's attribute information acquired at the procedure place.

### (Supplementary Note 9)

The information processing apparatus according to supplementary note 8, wherein the attribute information includes at least one of gender, age, and nationality.

### (Supplementary Note 10)

The information processing apparatus according to any one of supplementary notes 1 to 9, wherein the determination unit determines whether or not the user is allowed to board based on a predetermined weighting for each of the plurality of procedure places.

### (Supplementary Note 11)

The information processing apparatus according to any one of supplementary notes 1 to 9, wherein the determination unit determines whether or not the user is allowed to board the airplane based on average or deviation of the plurality of body surface temperatures.

### (Supplementary Note 12)

The information processing apparatus according to any one of supplementary notes 1 to 9, wherein the determination unit determines whether or not the user is allowed to board the airplane based on a maximum value of the plurality of body surface temperatures.

### (Supplementary Note 13)

The information processing apparatus according to any one of supplementary notes 1 to 9, wherein the determination unit determines whether or not the user is allowed to board the airplane based on a range of variation of the plurality of body surface temperatures.

### (Supplementary Note 14)

The information processing apparatus according to any one of supplementary notes 1 to 13, further comprising:
a control unit that causes an infrared capturing device installed at each of the plurality of procedure places to capture an infrared image,
wherein the acquisition unit acquires the body surface temperature based on the infrared image.

### (Supplementary Note 15)

An information processing method comprising:
acquiring body surface temperatures of a user of an airplane measured at procedure places of the plurality of procedures imposed on the user at a departure airport; and
determining whether the user is allowed to board the airplane based on the plurality of body surface temperatures acquired from the user.

### (Supplementary Note 16)

A storage medium storing a program that causes a computer to perform:
acquiring body surface temperatures of a user of an airplane measured at procedure places of a plurality of procedures imposed on the user at a departure airport; and
determining whether the user is allowed to board the airplane based on the plurality of body surface temperatures acquired from the user.

### REFERENCE SIGNS LIST

NW network
10 management server
10a token ID information DB
10b passage history information DB
10c operation information DB
20 check-in terminal
30 automatic baggage drop-off machine
40 security inspection apparatus
50 automated gate apparatus
60 boarding gate apparatus
65 operation terminal
100 information processing apparatus
100A acquisition unit
100B determination unit

What is claimed is:

1. An information processing apparatus comprising:
at least one memory storing instructions; and
at least one processor configured to execute the instructions to:
acquire body surface temperatures of a user of an airplane measured at of procedure places of a plurality of procedures imposed on the user at a departure airport; and
determine whether the user is allowed to board the airplane based on the plurality of body surface temperatures acquired from the user.

2. The information processing apparatus according to claim 1, wherein the at least one processor is further configured to execute the instructions to:
associate the registered face image of the user and the body surface temperature acquired at a procedure place in a check-in procedure with identification information that uniquely identifies the user; and
perform face matching between the registered face image and the user's face image acquired at the location of procedure whose order is later than the check-in procedure,
wherein the at least one processor associates the body surface temperature at the procedure place to the identification information when the user is authenticated by the face matching.

3. The information processing apparatus according to claim 1, wherein the at least one processor determines whether or not the user is allowed to board the airplane based on a result of comparison between the plurality of body surface temperatures and a predetermined reference value.

4. The information processing apparatus according to claim 3, wherein when there are a predetermined number of values is equal to or larger than the reference value among the plurality of the body surface temperatures, the at least one processor refuses the user to board the airplane.

5. The information processing apparatus according to claim 4, wherein the at least one processor counts the number of times when the body surface temperature of the user is equal to or higher than the reference value, and refuses the user to board the airplane at the procedure place where the number of times has reached the predetermined number.

6. The information processing apparatus according to claim 3, wherein the at least one processor refuses the user to board the airplane when a body surface temperature at a specific procedure place is equal to or higher than the reference value.

7. The information processing apparatus according to claim 3, wherein the at least one processor changes the reference value for each user based on the basal body temperature registered in advance for each user.

8. The information processing apparatus according to claim 3, wherein the at least one processor changes the reference value for each user based on the user's attribute information acquired at the procedure place.

9. The information processing apparatus according to claim 8, wherein the attribute information includes at least one of gender, age, and nationality.

10. The information processing apparatus according to claim 1, wherein the at least one processor determines whether or not the user is allowed to board based on a predetermined weighting for each of the plurality of procedure places.

11. The information processing apparatus according to claim 1, wherein the at least one processor determines whether or not the user is allowed to board the airplane based on average or deviation of the plurality of body surface temperatures.

12. The information processing apparatus according to claim 1, wherein the at least one processor determines whether or not the user is allowed to board the airplane based on a maximum value of the plurality of body surface temperatures.

13. The information processing apparatus according to claim 1, wherein the at least one processor determines whether or not the user is allowed to board the airplane based on a range of variation of the plurality of body surface temperatures.

14. The information processing apparatus according to claim 1, wherein the at least one processor is further configured to execute the instructions to:
cause an infrared capturing device installed at each of the plurality of procedure places to capture an infrared image,
wherein the at least one processor acquires the body surface temperature based on the infrared image.

15. An information processing method comprising:
acquiring body surface temperatures of a user of an airplane measured at procedure places of the plurality of procedures imposed on the user at a departure airport; and
determining whether the user is allowed to board the airplane based on the plurality of body surface temperatures acquired from the user.

16. A non-transitory storage medium storing a program that causes a computer to perform:
acquiring body surface temperatures of a user of an airplane measured at procedure places of a plurality of procedures imposed on the user at a departure airport; and
determining whether the user is allowed to board the airplane based on the plurality of body surface temperatures acquired from the user.
